SEQUENCE LISTING (6 sequences)

SEQ ID NO: 1 - DNA, 26 bp, Ovis aries
gtgctcggtgggcattcccacctcgg

SEQ ID NO: 2 - DNA, 27 bp, Ovis aries
ttccccttcccaggccagactcataga

SEQ ID NO: 3 - DNA, 15 bp, Ovis aries
tgctcggtgggcatt

SEQ ID NO: 4 - DNA, 19 bp, Ovis aries
ctcccatccctggtcttta

SEQ ID NO: 5 - DNA, 28 bp, Ovis aries
tccacacccatcctgctccagccactct

SEQ ID NO: 6 - DNA, 28 bp, Ovis aries
tgcagctgtctgcaagcaccccataccc

(19) United States
(12) Patent Application Publication
(10) Pub. No.: US 2026/0078453 A1
(43) Pub. Date: Mar. 19, 2026

(54) Methods and Reagents for Determining Desirable Meat Eating Quality Traits in Ovine Animals and Use of Same in Animal Breeding

(71) Applicant: Tattykeel Pty Ltd, Black Springs, New South Wales (AU)

(72) Inventors: Aduli MALAU-ADULI, Townsville (AU); Graham GILMORE, Black Springs (AU)

(21) Appl. No.: 18/879,629

(22) PCT Filed: Jun. 30, 2022
(86) PCT No.: PCT/AU2022/050685

§ 371 Date: Dec. 27, 2024

(51) Int. Cl. C12Q 1/6888; A01K 67/02

(52) U.S. Cl. CPC C12Q 1/6888; A01K 67/02; C12Q 2600/124; C12Q 2600/156

(57) ABSTRACT

The present disclosure relates generally to methods and test kits for identifying SNPs associated with desirable meat eating quality traits in ovine animals. In particular, the present disclosure relates to a SNP-based diagnostic test and method for identifying ovine animals with desirable fat melting point (FMP), intramuscular fat (IMF) and omega-3 long chain polyunsaturated fatty acids (n-3 LC PUFAs) which are characteristic of Australian White sheep or Lamb (e.g., Tattykeel Australian White Lamb), and the use of those tests and methods in animal breeding programs.

### TECHNICAL FIELD

The present disclosure relates generally to methods and test kits for identifying SNPs associated with desirable meat eating quality traits in ovine animals. In particular, the present disclosure relates to a SNP-based diagnostic test and method for identifying ovine animals with desirable fat melting point (FMP), intramuscular fat (IMF) and omega-3 long chain polyunsaturated fatty acids (n-3 LC PUFAs) which are characteristic of Australian White sheep or Lamb (e.g., Tattykeel Australian White Lamb), and the use of those tests and methods in animal breeding programs.

### BACKGROUND

Eating quality is the single largest determinant of consumer acceptability and satisfaction with meat products. The eating and nutritional quality of lamb is influenced by intramuscular fat (IMF) content (Thomas et. al., (2021). Animal, 15:100136), fat melting point (FMP), tenderness, juiciness, flavour (Pewan et. al., (2020) Antioxidants, 9:1118), and health-promoting omega-3 long-chain polyunsaturated fatty acids (n-3 LC-PUFA) that optimize retinal, maternal, and childhood brain functions while minimizing the risks associated with cardiovascular and chronic diseases (Heck et. al., (2021) Curr. Opin. Food Sci. 40:6-12; Pewan et. al., (2020) Genes, 11:587).

In a recent review of the development, calibration, and validation of objective measurement technologies for carcass composition, lean, fat, and meat-eating quality traits in the Australian and New Zealand livestock industries, Gardner et. al., (2021) Meat Sci. 179: 108556 highlighted the inherent difficulties associated with the poor measurement of meat-eating quality and lean meat yield. Attempts to predict IMF (Fowler et. al., (2021) Meat Sci. 177:108505; Lambe, et. al., (2021) Meat Sci. 17:108286; Alvarenga, et. al., (2021) Meat Sci. 181:108404), intramuscular connective tissue (Andueza et. al., (2021) Meat Sci., 179:108537), composition and quality characteristics (Patel et. al., (2021) Meat Sci., 178:108518), tenderness, ultimate pH, and IMF content (Dixit et. al (2020) Meat Sci., 162:108026020; Dixit et. al., (2021) Meat Sci., 181:108410; Knight et. al (2019) Meat Sci., 155:102-108) from near infra-red based regression equations were characterized by low accuracy, inconsistency, and divergence between calibration and validation data. Such inaccuracies lead to lamb inefficiencies and an estimated annual value-chain wastage costs of $130 million to the Australian beef industry (Gardner et. al., (2021) Meat Sci., 179:108556).

However, meat quality data can only be obtained after slaughter when selection decisions about the live animal are already too late. Carcass estimated breeding values (Knight et. al., (2020) Meat Sci., 170:108236; Anderson et. al., (2016) Meat Sci., 116:243-252), visual marbling score and meat imaging camera marbling systems (Stewart et. al., (2021) Meat Sci., 181:108369), and dual X-ray absorptiometry scanner based computed tomography determined fat, lean muscle, and bone compositions of lamb carcasses (Connaughton et. al., (2021) Meat Sci. 181:108413) are all useful technological advancements, but still present precision problems due to low accuracy, and by the time an informed decision on the genetic merit for meat quality is made, the animal is already dead. In a study of associations of sire estimated breeding values and objective meat quality measurements with sensory scores in Australian lamb, Pannier et al. (2014) Meat Sci., 96:1076-1087 confirmed the growing concerns that selecting for lean meat yield would reduce consumer eating quality and concluded that careful monitoring of selection programs is needed to maintain lamb eating quality. In an experimental trial to understand the impact of sire lean meat yield breeding value on carcass composition, meat quality, nutrient, and mineral content of Australian lamb, Knight et al., (2020) Meat Sci., 170:108236 concluded that to avoid deterioration in meat quality, the nutritional content of lamb and fresh meat color, Australian sheep producers will need to incorporate other aspects of meat quality when selecting sires with increased lean meat yield. To date, the use of conventional laboratory-based fat extraction, ‘slip point’ and gas chromatography methods still remain the most accurate techniques for not only measuring IMF, FMP, and n-3 LC-PUFA, but also for predicting consumer acceptance of beef and sheep meat (Holman et. al., (2021) Meat Sci., 181:108586). There is therefore a need for improved methods of measuring IMF, FMP, and n-3 LC-PUFA in livestock species.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY

The present disclosure is based, at least in part, on the recognition by the inventors that there is a need for improved methods for measuring IMF, FMP, and n-3 LC-PUFA in sheep species, and in particular in lambs bred for meat, to assist with genetics, breeding, and selection programs for meat-eating quality. To that end, the inventors combined (1) a minimally invasive longissimus dorsi thoracis et lumborum muscle biopsy sampling of Tattykeel Australian White (TAW) sheep, (2) laboratory based IMF, FMP, and fatty acid analyses, and (3) next-generation sequencing (NGS) of single nucleotide polymorphisms (SNP) within lipid metabolism genes, to directly quantifying the genetic worth of live lambs for health-beneficial n-3 LC-PUFA, IMF, and FMP.

Based on this work, the inventors identified several functional SNPs that are strongly associated with n-3 LC-PUFA, IMF, and FMP, as well as SNP markers which provide a unique DNA marker signature for TAW sheep genetics. Based on this finding, the inventors have developed a SNP-based test for identifying sheep with desirable meat-eating quality traits, including n-3 LC-PUFA, IMF and FMP, for use in animal breeding programs. The SNP-based test may also be used to determine and/or confirm that a sheep/lamb is a ‘true to type’ TAW sheep/lamb.

Accordingly, in one example, the present disclosure provides a method for identifying an ovine animal having desirable n-3 LC-PUFA, IMF, FMP or any combination thereof, as compared to the general population of animals of that species, said method comprising:
(a) obtaining single nucleotide polymorphism (SNP) genotype data for the ovine animal at one or more SNP loci selected from: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

In some examples, the method comprises:
(a) obtaining SNP genotype data for the ovine animal at SNP locus SCD g.23881050T>C or a SNP in linkage disequilibrium with SCD g.23881050T>C having an r² value of ≥0.7; and
(b) determining that the ovine animal has or will have desirable n-3 LC-PUFA, IMF, FMP or any combination thereof, as compared to the general population of animals of that species, if the animal possesses a TT genotype in SCD g.23881050T>C, or a genotype in linkage disequilibrium with said TT genotype.

For example, the method comprises:
(a) obtaining SNP genotype data for the ovine animal at SCD g.23881050T>C; and
(b) determining that the ovine animal has or will have desirable n-3 LC-PUFA, IMF, FMP or any combination thereof, as compared to the general population of animals of that species, if the animal possesses a TT genotype in SCD g.23881050T>C.

In some examples, the method comprises:
(a) obtaining SNP genotype data for the ovine animal at SNP locus FASN g.12323864A>G or a SNP in linkage disequilibrium with FASN g.12323864A>G having an r² value of ≥0.7; and
(b) determining that the ovine animal has or will have desirable n-3 LC-PUFA, IMF, FMP or any combination thereof, as compared to the general population of animals of that species, if the animal possesses a GG genotype in FASN g.12323864A>G, or a genotype in linkage disequilibrium with said GG genotype.

For example, the method comprises:
(a) obtaining SNP genotype data for the ovine animal at SNP locus FASN g.12323864A>G; and
(b) determining that the ovine animal has or will have desirable n-3 LC-PUFA, IMF, FMP or any combination thereof, as compared to the general population of animals of that species, if the animal possesses a GG genotype in FASN g.12323864A>G.

In some examples, the method comprises:
(a) obtaining SNP genotype data for the ovine animal at SNP locus FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses a AA genotype in FABP4 g.62829478A>T, or a genotype in linkage disequilibrium with said AA genotype.

For example, the method comprises:
(a) obtaining SNP genotype data for the ovine animal at SNP locus FABP4 g.62829478A>T; and
(b) determining that the ovine animal has or will have desirable n-3 LC-PUFA, IMF, FMP or any combination thereof, as compared to the general population of animals of that species, if the animal possesses a AA genotype in FABP4 g.62829478A>T.

In some examples, the method comprises:
(a) obtaining SNP genotype data for the ovine animal at two or three of the following SNP locations: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses one or more of the following SNP genotypes: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

In some examples, the ovine animal has or will have desirable n-3 LC-PUFA, IMF, FMP or any combination thereof, as compared to the general population of animals of that species, if the animal possesses one of the following SNP genotypes:
(i) a TT genotype in SCD g.23881050T>C or a genotype in linkage disequilibrium with said TT genotype and a GG genotype in FASN g.12323864A>G or a genotype in linkage disequilibrium with said GG genotype;
(ii) a TT genotype in SCD g.23881050T>C or a genotype in linkage disequilibrium with said TT genotype and a AA genotype in FABP4 g.62829478A>T or a genotype in linkage disequilibrium with said AA genotype;
(iii) a GG genotype in FASN g.12323864A>G or a genotype in linkage disequilibrium with said GG genotype and a AA genotype in FABP4 g.62829478A>T or a genotype in linkage disequilibrium with said AA genotype; and
(vi) a TT genotype in SCD g.23881050T>C or a genotype in linkage disequilibrium with said TT genotype, a GG genotype in FASN g.12323864A>G or a genotype in linkage disequilibrium with said GG genotype, and a AA genotype in FABP4 g.62829478A>T or a genotype in linkage disequilibrium with said AA genotype.

For example, the ovine animal has or will have desirable n-3 LC-PUFA, IMF, FMP or any combination thereof, as compared to the general population of animals of that species, if the animal possesses one of the following SNP genotypes:
(i) a TT genotype in SCD g.23881050T>C and a GG genotype in FASN g.12323864A>G;
(ii) a TT genotype in SCD g.23881050T>C and a AA genotype in FABP4 g.62829478A>T;
(iii) a GG genotype in FASN g.12323864A>G and a AA genotype in FABP4 g.62829478A>T;
(vi) a TT genotype in SCD g.23881050T>C, a GG genotype in FASN g.12323864A>G, and a AA genotype in FABP4 g.62829478A>T.

The method may further comprise selecting and/or breeding from an ovine animal identified as having desirable n-3 LC-PUFA, IMF, FMP or any combination thereof, as compared to the general population of animals of that species, based on SNP genotype.

In one example, the method identifies an ovine animal having desirable n-3 LC-PUFA as compared to the general population of animals of that species. In one example, the method identifies an ovine animal having desirable IMF as compared to the general population of animals of that species. In one example, the method identifies an ovine animal having desirable FMP as compared to the general population of animals of that species.

As described herein, the inventors have also identified SNP markers which provide a unique DNA marker signature for Tattykeel Australian White (TAW) sheep genetics. These markers may be used in a genetic test to determine and/or confirm that a sheep/lamb is a ‘true to type’ TAW sheep/lamb Accordingly, the present disclosure also provides a method for determining if an ovine animal or part thereof is an Australian White Lamb or part thereof, said method comprising:
(a) obtaining single nucleotide polymorphism (SNP) genotype data for the ovine animal at one or more SNP loci selected from: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7; and
(b) determining that the ovine animal or part thereof is an Australian White Lamb or part thereof if the animal possesses: (i) a TT genotype in SCD g.23881050T>C or a genotype in linkage disequilibrium with said TT genotype, (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

In some examples, the method comprises:
(a) obtaining SNP genotype data for the ovine animal at SNP locus SCD g.23881050T>C or a SNP in linkage disequilibrium with SCD g.23881050T>C having an r² value of ≥0.7; and
(b) determining that the ovine animal or part thereof is an Australian White Lamb or part thereof if the animal possesses a TT genotype in SCD g.23881050T>C, or a genotype in linkage disequilibrium with said TT genotype.

For example, the method comprises:
(a) obtaining SNP genotype data for the ovine animal at SCD g.23881050T>C; and
(b) determining that the ovine animal or part thereof is an Australian White Lamb or part thereof if the animal possesses a TT genotype in SCD g.23881050T>C.

In some examples, the method comprises:
(a) obtaining SNP genotype data for the ovine animal at SNP locus FASN g.12323864A>G or a SNP in linkage disequilibrium with FASN g.12323864A>G having an r² value of ≥0.7; and
(b) determining that the ovine animal or part thereof is an Australian White Lamb or part thereof if the animal possesses a GG genotype in FASN g.12323864A>G, or a genotype in linkage disequilibrium with said GG genotype.

For example, the method comprises:
(a) obtaining SNP genotype data for the ovine animal at SNP locus FASN g.12323864A>G; and
(b) determining that the ovine animal or part thereof is an Australian White Lamb or part thereof if the animal possesses a GG genotype in FASN g.12323864A>G.

In some examples, the method comprises:
(a) obtaining SNP genotype data for the ovine animal at SNP locus FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses a AA genotype in FABP4 g.62829478A>T, or a genotype in linkage disequilibrium with said AA genotype.

For example, the method comprises:
(a) obtaining SNP genotype data for the ovine animal at SNP locus FABP4 g.62829478A>T; and
(b) determining that the ovine animal has or will have desirable n-3 LC-PUFA, IMF, FMP or any combination thereof, as compared to the general population of animals of that species, if the animal possesses a AA genotype in FABP4 g.62829478A>T.

In some examples, the method comprises:
(a) obtaining SNP genotype data for the ovine animal at two or three of the following SNP locations: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7; and
(b) determining that the ovine animal or part thereof is an Australian White Lamb or part thereof if the animal possesses one or more of the following SNP genotypes: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

In some examples, the ovine animal is determined as being an Australian White Lamb or part thereof if the animal possesses one of the following genotypes:
(i) a TT genotype in SCD g.23881050T>C or a genotype in linkage disequilibrium with said TT genotype and a GG genotype in FASN g.12323864A>G or a genotype in linkage disequilibrium with said GG genotype;
(ii) a TT genotype in SCD g.23881050T>C or a genotype in linkage disequilibrium with said TT genotype and a AA genotype in FABP4 g.62829478A>T or a genotype in linkage disequilibrium with said AA genotype;
(iii) a GG genotype in FASN g.12323864A>G or a genotype in linkage disequilibrium with said GG genotype and a AA genotype in FABP4 g.62829478A>T or a genotype in linkage disequilibrium with said AA genotype; and
(vi) a TT genotype in SCD g.23881050T>C or a genotype in linkage disequilibrium with said TT genotype, a GG genotype in FASN g.12323864A>G or a genotype in linkage disequilibrium with said GG genotype, and a AA genotype in FABP4 g.62829478A>T or a genotype in linkage disequilibrium with said AA genotype.

For example, the ovine animal is determined as being an Australian White Lamb or part thereof if the animal possesses one of the following genotypes:
(i) a TT genotype in SCD g.23881050T>C and a GG genotype in FASN g.12323864A>G;
(ii) a TT genotype in SCD g.23881050T>C and a AA genotype in FABP4 g.62829478A>T;
(iii) a GG genotype in FASN g.12323864A>G and a AA genotype in FABP4 g.62829478A>T;
(vi) a TT genotype in SCD g.23881050T>C, a GG genotype in FASN g.12323864A>G, and a AA genotype in FABP4 g.62829478A>T.

The method may further comprise selecting and/or breeding from an ovine animal or part thereof identified as being an Australian White Lamb based on SNP genotype.

In each of the foregoing examples, the method may further comprise generating the SNP genotype data from a nucleic acid sample from the ovine animal. Any method known in the art for generating SNP genotype data from a nucleic acid sample is contemplated. Exemplary methods are described herein.

The nucleic acid sample may be obtained or prepared from any suitable biological sample obtained from the ovine animal, such as a tissue, fibre or fluid (e.g., blood, blood fraction or semen) or combinations thereof. In some examples, the nucleic acid sample is prepared from tissue (e.g., muscle) taken from the longissimus dorsi thoracis et lumborum of the animal. In other examples, the nucleic acid sample is prepared from reproductive material of an animal selected from semen, an oocyte or an embryo. In other examples, the nucleic acid sample is prepared from fibre obtained from an animal or cells attached thereto (e.g., wool fibre or follicle cells attached thereto). In other examples, the nucleic acid sample is prepared from blood obtained from an animal (e.g., whole blood or a blood fraction such as serum). In each of the foregoing examples, the nucleic acid sample may be DNA. However, in some examples it may be desirable to prepare RNA from the animal.

As described herein, any method known in the art for generating SNP genotype data from a nucleic acid sample is contemplated. However, in some examples, the SNP genotype data is generated using one or more assays selected from the group consists of a DNA amplification assay, a DNA hybridisation assay, DNA sequencing, denaturing high-performance liquid chromatography (DHPLC) or a combination thereof. In one example, a DNA amplification is employed in the method. For example, SNP alleles may detected by one or more DNA amplification assays using polynucleotides which permit different alleles of a SNP to be distinguished from one another. Exemplary DNA amplification assays for use in the method of the disclosure are described herein. However, in one example, the DNA amplification assay is polymerase chain reaction (PCR) e.g., amplification-refractory mutation system (ARMS) PCR.

In certain examples, the method may comprise detection of SNP alleles by one or more DNA hybridisation assays using polynucleotides which permit different alleles of a SNP to be distinguished from one another.

In other examples, the method may comprise detection of SNP alleles for each ovine animal by sequencing a gene or subsequence of the gene comprising the or each SNP. Exemplary DNA sequencing methods which are contemplated for use in the method of the disclosure are described herein.

The present disclosure also provides a system for determining the nucleotide occurrence at one or more ovine SNPs selected from SCD g.23881050T>C, FASN g.12323864A>G and FABP4 g.62829478A>T. The system may comprise a hybridisation medium and/or solid substrate that comprises at least two oligonucleotides, each configured to hybridise selectively to a specific allele of an ovine SNP selected from SCD g.23881050T>C, FASN g.12323864A>G and FABP4 g.62829478A>T. For example, where the system is for determining the nucleotide occurrence at two or more (e.g., three) ovine SNPs selected from SCD g.23881050T>C, FASN g.12323864A>G and FABP4 g.62829478A>T, the hybridisation medium and/or substrate comprises at least two oligonucleotides for each SNP, wherein each oligonucleotide is configured to hybridise selectively to a specific allele of the respective ovine SNP. For example, a solid support may be provided, to which the oligonucleotides are directly or indirectly attached.

The present disclosure also provides a test kit comprising a plurality of oligonucleotide primers and/or probes configured to discriminate between alleles at one or more SNP loci in an ovine animal, wherein the SNP loci are selected from (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or FABP4 g.62829478A>T or a SNP in linkage disequilibrium with FABP4 g.62829478A>T having an r² value of ≥0.7.

In one example, the test kit may comprise:
one or more oligonucleotide primers and/or probes configured to discriminate between allele C and allele T at SNP locus SCD g.23881050T>C;
one or more oligonucleotide primers and/or probes configured to discriminate between allele G and allele A at SNP locus FASN g.12323864A>G; and/or
one or more oligonucleotide primers and/or probes configured to discriminate between allele A and allele T at SNP locus FABP4 g.62829478A>T.

In one example, the test kit comprises one or more oligonucleotide primers and/or probes configured to discriminate between allele C and allele T at SNP locus SCD g.23881050T>C. For example, the oligonucleotide primers may be configured to discriminate between allele C and allele T at SNP locus SCD g.23881050T>C by amplification-refractory mutation system polymerase chain reaction (ARMS PCR). In a particular example, the oligonucleotide primers which are configured to discriminate between allele C and allele T at SNP locus SCD g.23881050T>C by ARMS PCR are:
SCD g.23881050T>C_Forward inner (SEQ ID NO: 1);
SCD g.23881050T>C_Reverse inner (SEQ ID NO: 2);
SCD g.23881050T>C_Forward outer (a) (SEQ ID NO: 3);
SCD g.23881050T>C_Reverse outer (a) (SEQ ID NO: 4);
SCD g.23881050T>C_Forward outer (b) (SEQ ID NO: 5); and
SCD g.23881050T>C_Reverse outer (b) (SEQ ID NO: 6).

The test kit may also comprise one or more or each of the following:
at least one DNA polymerase enzyme;
dinucleotide triphosphates (dNTPs);
a magnesium salt; and
a buffer.

The test kit may be used in a method of the disclosure. For example, the test kit may be used in a method as described herein for identifying or determining an ovine animal having desirable n-3 LC-PUFA, intramuscular fat (IMF), fat melting point (FMP) or any combination thereof, as compared to the general population of animals of that species. Alternatively, or in addition, the test kit may be used in a method as described herein for determining if an ovine animal or part thereof is an Australian White Lamb or part thereof.

The present disclosure also provides a method of producing reproductive or regenerative material from an ovine animal determined as having desirable n-3 LC-PUFA, IMF, FMP or any combination thereof, as compared to the general population of animals of that species, comprising:
(i) performing the method as described herein on one or more animals to thereby identify an ovine animal having desirable n-3 LC-PUFA, IMF, FMP or any combination thereof, as compared to the general population of animals of that species;
(ii) selecting the animal identified in (i) as having desirable n-3 LC-PUFA, IMF, FMP or any combination thereof, as compared to the general population of animals of that species; and
(iii) obtaining reproductive or regenerative material from the selected animals.

The present disclosure also provides a method of producing reproductive or regenerative material from an ovine animal determined as having a SNP genotype indicative of AWL genetics, comprising:
(i) performing the method as described herein on one or more animals to thereby identify an ovine animal having a SNP genotype indicative of AWL genetics;
(ii) selecting the animal identified in (i) as having a SNP genotype indicative of AWL genetics; and
(iii) obtaining reproductive or regenerative material from the selected animals;
wherein the SNP genotype indicative of AWL genetics comprises (i) a TT genotype in SCD g.23881050T>C or a genotype in linkage disequilibrium with said TT genotype, (ii) a GG genotype in FASN g.12323864A>G or a genotype in linkage disequilibrium with said GG genotype, and/or (iii) a AA genotype in FABP4 g.62829478A>T or a genotype in linkage disequilibrium with said AA genotype. In some examples, the SNP genotype indicative of AWL genetics comprises one or more of a TT genotype in SCD g.23881050T>C, a GG genotype in FASN g.12323864A>G or a AA genotype in FABP4 g.62829478A>T. In one particular example, the SNP genotype indicative of AWL genetics comprises a TT genotype in SCD g.23881050T>C.

The present disclosure also provides reproductive or regenerative material from an ovine animal produced according to the method described herein.

The present disclosure also provides a method of breeding an ovine animal having desirable n-3 LC-PUFA, IMF, FMP or any combination thereof, as compared to the general population of animals of that species, comprising:
(i) performing the method as described herein on one or more animals to thereby identify one or more ovine animals having desirable n-3 LC-PUFA, IMF, FMP or any combination thereof, as compared to the general population of animals of that species;
(ii) selecting one or more animals identified in (i) as having desirable n-3 LC-PUFA, IMF, FMP or any combination thereof, as compared to the general population of animals of that species;
(iii) breeding from the selected animals to produce one or more offspring therefrom.

The present disclosure also provides an ovine animal bred according to the method described herein.

The present disclosure also provides a method of selectively breeding an Australian White Lamb (AWL), comprising:
(i) performing the method as described herein on one or more animals to thereby identify one or more ovine animals having a SNP genotype indicative of AWL genetics;
(ii) selecting one or more animals identified in (i) as having a SNP genotype indicative of AWL genetics;
(iii) breeding from the selected animals to produce one or more offspring therefrom;
wherein the SNP genotype indicative of AWL genetics comprises: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype. In some examples, the SNP genotype indicative of AWL genetics comprises one or more of a TT genotype in SCD g.23881050T>C, a GG genotype in FASN g.12323864A>G and/or a AA genotype in FABP4 g.62829478A>T. In one particular example, the SNP genotype indicative of AWL genetics comprises one or more of a TT genotype in SCD g.23881050T>C.

The present disclosure also provides an ovine animal bred according to the method described herein.

The present disclosure also provides a method for improving meat eating quality in a population of ovine animals, comprising:
(i) performing the method as described herein on one or more animals in the population to thereby identify one or more ovine animals having desirable n-3 LC-PUFA, IMF, FMP or any combination thereof, as compared to the general population of animals of that species;
(ii) selecting one or more animals identified in (i) as having desirable n-3 LC-PUFA, IMF, FMP or any combination thereof, as compared to the general population of animals of that species;
(iii) obtaining reproductive or regenerative material from the selected animals; and
(iv) producing one or more offspring or one or more generations of animals from the reproductive or regenerative material.

In each of the foregoing examples, the reproductive material may be an oocyte, an embryo and/or semen. Similarly, regenerative material may be an oocyte, an embryo and/or semen, but may further includes primordial germ cells (PGCs) that give rise to oocytes or spermatozoa.

### BRIEF DESCRIPTION OF THE DRAWINGS

FIG. 1. Experimental design for the selection, breeding, and evaluation of n-3 LC-PUFA, IMF, and FMP in Tattykeel Australian White sheep.

FIG. 2. FASN fragment 1 PCR product in Tattykeel Australian White (WL), Poll Dorset (PD), and Texel (TX) lambs.

FIG. 3. FASN fragment 2 PCR product in Tattykeel Australian White (WL), Poll Dorset (PD), and Texel (TX) lambs.

FIG. 4. FASN fragment 3 PCR product in Tattykeel Australian White (WL), Poll Dorset (PD), and Texel (TX) lambs.

FIG. 5. SCD PCR product in Tattykeel Australian White (WL), Poll Dorset (PD), and Texel (TX) lambs.

FIG. 6. FABP4 PCR product of Tattykeel Australian White (WL), Poll Dorset (PD), and Texel (TX) lambs.

FIG. 7. Correlations between SCD gene SNP loci, IMF, FMP, and fatty acids in TAW lambs.

FIG. 8. Correlations between FASN gene SNP loci, IMF, FMP, and fatty acids in TAW lambs.

FIG. 9. Correlations between FABP4 gene SNP loci, IMF, FMP, and fatty acids in TAW lambs.

FIG. 10. TAWL, Texel and Poll Dorset (10 uM tetra-primers with forward outer/reverse outer primer (a) and 0.5 uL gDNA).

FIG. 11. TAWL, Texel and Poll Dorset (100 uM of forward outer Primer (a), 10 uM forward inner/reverse inner and reverse outer primers (a), 0.5 uL gDNA).

FIG. 12. TAWL, Texel and Poll Dorset (10 uM tetra-primers with forward outer/reverse outer primer (b) and 0.5 uL gDNA).

FIG. 13. TAWL, Wagyu, Angus, Hereford and Brahman (10 uM tetra-primers with forward outer/reverse outer primer (b) and 0.5 uL gDNA).

### KEY TO THE SEQUENCE LISTING

SEQ ID NO:1: Oligonucleotide sequence for primer SCD g.23881050T>C_Forward inner
SEQ ID NO:2: Oligonucleotide sequence for primer SCD g.23881050T>C_Reverse inner
SEQ ID NO:3: Oligonucleotide sequence for primer SCD g.23881050T>C_Forward outer (a)
SEQ ID NO:4: Oligonucleotide sequence for primer SCD g.23881050T>C_Reverse outer (a)
SEQ ID NO:5: Oligonucleotide sequence for primer SCD g.23881050T>C_Forward outer (b)
SEQ ID NO:6: Oligonucleotide sequence for primer SCD g.23881050T>C_Reverse outer (b)

### DETAILED DESCRIPTION

### General Techniques and Selected Definitions

Unless specifically defined otherwise, all technical and scientific terms used herein shall be taken to have the same meaning as commonly understood by one of ordinary skill in the art.

As used herein, the singular forms of “a”, “and” and “the” include plural forms of these words, unless the context clearly dictates otherwise.

The term “and/or”, e.g., “X and/or Y” shall be understood to mean either “X and Y” or “X or Y” and shall be taken to provide explicit support for both meanings or for either meaning.

Throughout this specification, the word “comprise” or variations such as “comprises” or “comprising” will be understood to imply the inclusion of a stated element, integer or step, or group of elements, integers or steps, but not the exclusion of any other element, integer or step, or group of elements, integers or steps.

The term “about” is used herein to mean approximately. When the term “about” is used in conjunction with a numerical range, it modifies that range by extending the boundaries above and below the recited numerical values. In general, the term “about” is used herein to modify a numerical value, such as an amount of time, concentration, temperature etc., above and below the stated value by ±10%, more preferably ±5%, even more preferably ±1%, and still more preferably ±0.1% from the specified value as appropriate to perform the disclosed method.

Those skilled in the art will appreciate that the present disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations of any two or more of said steps or features. Thus, each feature of any particular aspect or embodiment of the present disclosure may be applied mutatis mutandis to any other aspect or embodiment of the present disclosure.

The present disclosure is not to be limited in scope by the specific embodiments described herein, which are intended for the purpose of exemplification only. Functionally equivalent products, compositions and methods are clearly within the scope of the disclosure, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The present disclosure as described herein can be performed using, unless otherwise indicated, conventional techniques of molecular biology. Such procedures are described, for example, in Sambrook, Fritsch & Maniatis, Molecular Cloning: A Laboratory Manual, Cold Spring Harbor Laboratories, New York, Second Edition (1989), whole of vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wunsch, E., ed. (1974) Synthese von Peptiden in Houben-Weyls Metoden der Organischen Chemie (Muler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, VoIs. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

### Methods of Animal Selection

The inventors have identified several functional SNPs within lipid metabolism genes that are strongly associated with n-3 LC-PUFA, IMF, and FMP, and which provide a unique DNA marker signature for TAW sheep genetics. Based on this finding, the inventors have developed a SNP-based test for identifying sheep with desirable meat-eating quality traits, including n-3 LC-PUFA, IMF and FMP, for use in animal breeding programs. The SNP-based test may also be used to determine and/or confirm that a sheep/lamb is (or is not) a ‘true to type’ TAW sheep/lamb e.g., for breeding purposes.

Accordingly, in one example, the present disclosure provides a method for identifying an ovine animal having desirable n-3 LC-PUFA, IMF, FMP or any combination thereof, as compared to the general population of animals of that species, said method comprising:
(a) obtaining single nucleotide polymorphism (SNP) genotype data for the ovine animal at one or more SNP loci selected from: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

As described herein, the method of the disclosure is useful for identifying an ovine animal having desirable n-3 LC-PUFA, IMF, FMP or any combination thereof. It has been shown that consumers prefer meat with low FMP, moderate IMF and fatty acid composition with proportionately more of the health-promoting n-3 LC-PUFA. Thus, the method of disclosure may be used to select for animals whose meat has a low FMP, moderate IMF and a fatty acid composition with proportionately more of the health-promoting n-3 LC-PUFA.

The term “fat melting point (FMP)” as used herein refers to the temperature at which the solid and liquid phases of fat are in equilibrium. The melting point is determined by fatty acid composition, the molecular structure of those fatty acids, and the way those fatty acids are ‘packed together’, among other things. Fatty acids are monocarboxylic acids composed of an aliphatic chain containing 4 to 22 carbon atoms with a terminal carboxyl group (COOH). The fatty acid can be saturated or unsaturated, branched or unbranched, and may or may not include one or more hydroxyl group(s). A low fat melting point is desirable for eating quality of meat. Fat melting point (or FMP) in sheep can range from about 25° C. to about 50° C., depending on sheep breed and genetics. In accordance with the method of the disclosure, a desirable FMP is one which is below 40° C. In one example, a desirable FMP is one which is between 28° C. to 39° C. (e.g., about 28° C., or about 29° C., or about 30° C., or about 31° C., or about 32° C., or about 33° C., or about 34° C., or about 35° C., or about 36° C., or about 37° C., or about 38° C., or about 39° C.). In a particular example, a desirable FMP is one which is, on average, between about 30° C. to 36° C. For example, animals selected as having desirable FMP in accordance with the method of the disclosure may have a FMP which is, on average, about 32° C. to 36° C. (e.g., about 34° C.). In one example, the method comprises identifying an ovine animal having a desirable FMP as compared to the general population of animals of that species.

“Intramuscular fat (IMF)” as used herein refers to fat which is located between and within muscle fibres (cells) and is often referred to as “marbling”. There is an indirect relationship between IMF and meat tenderness, juiciness, flavour and appearance. A number of methods are available for measuring IMF, and each of these is contemplated herein. However, perhaps the most practical and reproducible method used for measuring IMF in a live animals is ultrasound. A “desirable” level of IMF as described herein is at least about 2.5% to about 10% (e.g., about 2.5%, or about 3.0%, or about 3.5%, or about 4.0%, or about 4.5%, or about 5.0%, or about 5.5%, or about 6.0%, or about 6.5%, or about 7.0%, or about 7.5%, or about 8.0%, or about 8.5%, or about 9.0%, or about 9.5%, or about 10.0%). In some examples, a “desirable” level of IMF is about 3.5% to about 8.5%. More preferably, a “desirable” level of IMF may be about 4.0% to 6.0%. In a particular example, the percentage of IMF which is desirable is (on average) about 4.4%. In one example, the method comprises identifying an ovine animal having a desirable level of IMF as compared to the general population of animals of that species.

As described herein, the method may be used to identify animals which have a desirable level of health-promoting omega-3 long-chain polyunsaturated fatty acids (n-3 LC-PUFA). As used herein, the term “n-3 LC-PUFA” or “omega-3 long-chain polyunsaturated fatty acids” refers to polyunsaturated fatty acids (PUFA) in which the ultimate ethylenic bond is 3 carbons from and including the terminal methyl group of the fatty acid. n-3 LC-PUFA is a known health-promoting fatty acid that has been shown to optimize retinal, maternal, and childhood brain functions while minimizing the risks associated with cardiovascular and chronic diseases. Exemplary n-3 LC-PUFA's include alpha linolenic acid (C18:3; “ALA”), eicosapentaenoic acid (C20:5; “EPA”) and docosahexaenoic acid (C22:6; “DHA”). Other n-3 LC-PUFAs include, but are not limited to, linoleic acid (C18:2n-6), palmitoleic acid (C16:0), stearic acid (C18:0), trans-9-octadecenoic acid (C18:1n-9), stearidonic acid (C18:4), eicosatetraenoic acid (C20:4), and docosapentaenoic acid (C22:5). The term “desirable” in the context of n-3 LC-PUFA shall therefore be understood to mean a desirable level or concentration of n-3 LC-PUFA in the meat of the animal. A desirable level or concentration of n-3 LC-PUFA will be at least about 40.0 mg/100 g of meat, such as between about 40.0 mg/100 g to about 150.0 mg/100 g of meat. For example, a desirable level of n-3 LC-PUFA may be about 40.0 mg/100 g, or about 50.0 mg/100 g, or about 60.0 mg/100 g, or about 70.0 mg/100 g, or about 80.0 mg/100 g, or about 90.0 mg/100 g, or about 100.0 mg/100 g, or about 110.0 mg/100 g, or about 120.0 mg/100 g, or about 130.0 mg/100 g, or about 140.0 mg/100 g, or about 150.0 mg/100 g of meat). In some examples, a desirable level of n-3 LC-PUFA is about 45.0 mg/100 g to about 120.0 mg/100 g of meat. In some examples, a desirable level of n-3 LC-PUFA is about 50.0 mg/100 g to about 100.0 mg/100 g of meat. In one example, a desirable level of n-3 LC-PUFA is about 55.0 mg/100 g to 60.0 mg/100 g. In a particular example, a desirable level of n-3 LC-PUFA is (on average) about 57.9 mg/100 g of meat. In one example, the method comprises identifying an ovine animal having a desirable level of n-3 LC-PUFA as compared to the general population of animals of that species.

The term “general population” as used in the context of ovine animals shall be understood to mean a plurality of ovine animals which, when considered as a population or group, are heterozygous at one or more of the SNP loci described herein i.e., one or more SNP loci located in selected from: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or (iii) FABP4 g.62829478A>T or a SNP in linkage disequilibrium with FABP4 g.62829478A>T having an r² value of ≥0.7. In some examples, the general population may not have undergone selective breeding following identification of animals having desirable n-3 LC-PUFA, IMF, FMP or any combination thereof according to the method described herein. In other examples, the general population may have undergone selective breeding following identification of animals having desirable n-3 LC-PUFA, IMF, FMP or any combination thereof according to the method described herein, but still comprises animals which are heterozygous at one or more of the SNP loci described herein or which are homozygous for alleles at the one or more of the SNP loci described herein which do not confer desirable n-3 LC-PUFA, IMF and/or FMP. That is, the population comprises genetic diversity at the SNP loci described herein. The general population of ovine animals may include male and/or female animals, and may include animals of any age and any breed. However, in particular examples, the general population predominantly comprises sheep which are a meat breed of sheep or a dual-purpose breed of sheep. The general population may comprise any number of animals, such as 2 animals or 1000 or more animals.

The present disclosure also provides a method for determining if an ovine animal or part thereof is an Australian White Lamb or part thereof, said method comprising:
(a) obtaining single nucleotide polymorphism (SNP) genotype data for the ovine animal at one or more SNP loci selected from: (i) SCD g.23881050T>C or a SNP in linkage disequilibrium with SCD g.23881050T>C having an r² value of ≥0.7; (ii) FASN g.12323864A>G or a SNP in linkage disequilibrium with FASN g.12323864A>G having an r² value of ≥0.7; and/or FABP4 g.62829478A>T or a SNP in linkage disequilibrium with FABP4 g.62829478A>T having an r² value of ≥0.7; and
(b) determining that the ovine animal or part thereof is an Australian White Lamb or part thereof if the animal possesses: (i) a TT genotype in SCD g.23881050T>C or a genotype in linkage disequilibrium with said TT genotype; (ii) a GG genotype in FASN g.12323864A>G or a genotype in linkage disequilibrium with said GG genotype; and/or (iii) a AA genotype in FABP4 g.62829478A>T or a genotype in linkage disequilibrium with said AA genotype.

As used herein, the term “ovine animal” refers to a sheep. The sheep may be male or female and of any age. The ovine animal may also be any breed of sheep. However, the method of the disclosure is particularly useful with meat breeds and dual-purpose breeds. In some examples, the ovine animal with which the method of the disclosure is used is a breed of meat sheep. Exemplary breeds of sheep that tend to be used for meat production include, but are not limited to, Australian White (also referred to as an Australian White Lamb (AWL) or Australian White Sheep), Poll Dorset, Suffolk, White Suffolk and Dorper. In other examples, the ovine animal with which the method of the disclosure is used is a dual-purpose sheep breed (i.e., a breed of sheep used for meat and fibre). Exemplary dual purpose breeds include, but are not limited to Border Leicester, Corriedale, Coopworth, Texel and South African Meat Merino (SAMM).

The term “Australian White” as used herein in the context of sheep or lamb i.e., “Australian White Sheep” “Australian White Lamb”, “AWL” or similar, refers to a breed of meat sheep developed in Australia. The terms “Australian White”, “Australian White Sheep” “Australian White Lamb” and “AWL” are used herein interchangeably. The breed was developed through selective breeding of White Dorper, Van Rooy, Poll Dorset and Texel sheep, with the aim of creating a large white sheep suited to Australian conditions, and with a self-shedding hair coat. This breed produces hardy, large framed, heavy weight lambs that reach slaughter weights early. This breed also has high reproductive capabilities, with early maturing and an open breeding season, making it a self-replacing flock.

The term “part thereof” as used in the context of an Australian White Lamb refers to a portion of an animal determined to be an Australian White Lamb. In some examples, the “part thereof” in the context of an Australian White Lamb may be a dressed carcass or a cut of meat obtained from an animal determined to be an Australian White Lamb. In another example, a “part thereof” in the context of an Australian White Lamb may be a biological sample, such as a tissue sample, fibre sample, blood sample or semen sample. Other exemplary biological samples are described herein and contemplated. In other examples, the “part thereof” may be reproductive material obtained from an animal determined to be an Australian White Lamb. Exemplary reproductive materials are described herein, but may include oocytes, embryos and/or semen.

To date, assessment of meat quality (including meat-eating quality) typically takes place post-slaughter. This can make it difficult to incorporate data on meat eating quality into breeding decisions. As discussed herein, the present disclosure is based on the identification of specific SNPs within the lipid metabolism genes SCD, FASN, and FABP4 which the inventors have shown to be associated with particular meat-eating quality traits, namely n-3 LC-PUFA, IMF, and/or FMP. These SNPs therefore make it possible to predict whether an animal is likely to have desirable n-3 LC-PUFA, IMF, and FMP, and thereby make early, informed decisions on the breeding value of live sheep in terms of meat-eating quality traits. It has also been identified that these SNP markers provide a unique DNA marker signature for Tattykeel Australian White (TAW) sheep genetics. These SNP markers may be used in a genetic test to determine and/or confirm that a sheep/lamb is a ‘true to type’ Australian White lamb, in particular a TAW sheep/lamb.

The terms “single nucleotide polymorphism” or “SNP” as used herein refer to a variation (“polymorphism”) at a single nucleotide position within a genome. Specifically, a SNP is a germline substitution of a single nucleotide at a specific position in the genome which is present in sufficiently large fraction of the population of an organism (e.g., 1% or more). For example, where a portion of a population possess an ‘A’ at a particular nucleotide position, and another portion of the population possesses a ‘C’, ‘G’ or ‘T’ at the same specific position within the genome, the nucleotide position constitutes a SNP. Different SNP variants e.g., an ‘A’, ‘C’, ‘G’ or ‘T’ at a particular nucleotide position, are referred to as an “allele”. Where an organism is a diploid species, as in the case of sheep, the animal will possess two copies of the polynucleotide harbouring the SNP, and therefore possess two SNP alleles. Collectively, SNP alleles at a particular SNP location are referred to as a SNP “genotype”. However, a “genotype” may refer to the genetic information an individual carries at one or more SNP positions in the genome. Where a diploid animal possesses two copies of the same SNP allele, e.g., AA, GG, TT or CC, the animal is said to be “homozygous” for the SNP. By contrast, where an animal possesses non-identical SNP alleles at a SNP location on respective homologous chromosomes e.g., AG, AC, AT, CG, CT or GT, then the animal is said to be “heterozygous” at the particular SNP.

As described herein, the SNPs associated with desirable n-3 LC-PUFA, IMF, and/or FMP may be selected from: (i) SCD g.23881050T>C; (ii) FASN g.12323864A>G; and/or (iii) FABP4 g.62829478A>T. These SNP locations have been defined by reference to the following coding sequences in the National Center for Biotechnology Information (NCBI) database (Genbank): SCD (NC_040273.1), FASN gene (Accession Number: NC_040262.1), FABP4 (NC_040260.1). In this regard, the inventors have shown that (i) SCD g.23881050T>C is associated with IMF, and n-3 LC-PUFAs C22:6n-3 (docosahexaenoic acid) and C22:5n-3 (docosapentanoic acid); (ii) FASN g.12323864A>G is associated with FMP, long chain saturated fatty acids C18:3n-3 (alpha linoleic acid), C18:1n-9 (elaidic acid), C18:0 (stearic acid), C16:0 (palmitic acid), and monounsaturated fatty acids, and (iii) FABP4 g.62829478A>T is associated with IMF. Accordingly, the method of the disclosure may comprise interrogating an animal's genotype at one or more of SCD g.23881050T>C, FASN g.12323864A>G, and FABP4 g.62829478A>T. In one example, a TT genotype in SCD g.23881050T>C is associated with desirable IMF and n-3 LC-PUFAs as compared to a TA or TT genotype in SCD g.23881050T>C. In one example, a GG genotype in FASN g.12323864A>G is associated with desirable FMP, long chain saturated fatty acids and monounsaturated fatty acids, as compared to a GA or AA genotype in FASN g.12323864A>G. In one example, an AA genotype in FABP4 g.62829478A>T is associated with IMF as compared to a AG or GG genotype in FABP4 g.62829478A>T.

The inventors have also determined that the above-mentioned SNP genotypes which are useful in predicting desirable n-3 LC-PUFA, IMF, and/or FMP may be used to determine if a sheep or lamb is a ‘true to type’ Australian White lamb, in particular a ‘true to type’ Tattykeel Australian White (TAW) sheep/lamb.

In other examples, the method of the disclosure may comprises making a selection decision on the basis of one or more SNPs or other polymorphic genetic markers which are in linkage disequilibrium (LD) with SCD g.23881050T>C, FASN g.12323864A>G, or FABP4 g.62829478A>T. This is based on the fact that other SNPs or polymorphic genetic markers in close proximity to SCD g.23881050T>C, FASN g.12323864A>G, or FABP4 g.62829478A>T will also be associated with desirable n-3 LC-PUFA, IMF, and/or FMP because markers in linkage disequilibrium with SCD g.23881050T>C, FASN g.12323864A>G, or FABP4 g.62829478A>T will also be in linkage disequilibrium with the gene(s) influencing n-3 LC-PUFA, IMF, and/or FMP.

“Linkage disequilibrium” or “LD” as used herein refers to the non-random association of alleles at two or more loci within a particular population. Said another way, linkage disequilibrium is the preferential association of a particular allele or genetic marker with a specific allele, or genetic marker at a nearby chromosomal location more frequently than expected by chance for any particular allele frequency in the population. Thus, linkage disequilibrium may be measured as a departure from the null hypothesis of linkage equilibrium, where each allele at one locus associates randomly with each allele at a second locus in a population of individual genomes. For example, if locus X has alleles A and B, which occur equally frequently, and linked locus Y has alleles C and D, which occur equally frequently, one would expect the combination AC to occur with a frequency of 0.25. If AC occurs more frequently, then alleles A and C are in linkage disequilibrium. Linkage disequilibrium may result from natural selection of certain combination of alleles or because an allele has been introduced into a population too recently to have reached equilibrium with linked alleles. For biallelic markers, one of the most commonly used measures for LD is r², the square of the correlation coefficient between two indicator variables—one representing the presence or absence of a particular allele at the first locus and the other representing the presence or absence of a particular allele at the second locus.

As used herein, the term “locus” or “loci” refers to the site(s) or location(s) of one or more genes or polynucleotide sequences on a chromosome.

In some examples, a SNP or other polymorphic marker (also collectively referred to as a “genetic marker”) which is in LD with SCD g.23881050T>C, FASN g.12323864A>G, or FABP4 g.62829478A>T may be used in lieu of SCD g.23881050T>C, FASN g.12323864A>G, or FABP4 g.62829478A>T respectively to predict the likelihood that an animal will have desirable n-3 LC-PUFA, IMF, and/or FMP. Similarly, a genetic marker which is in LD with SCD g.23881050T>C, FASN g.12323864A>G, or FABP4 g.62829478A>T may be used in lieu of SCD g.23881050T>C, FASN g.12323864A>G, or FABP4 g.62829478A>T respectively to determine if a sheep or lamb is a ‘true to type’ Australian White lamb, in particular a ‘true to type’ Tattykeel Australian White (TAW) sheep/lamb.

More particularly, the method of the disclosure may comprise determining that an ovine animal has or will have desirable n-3 LC-PUFA, IMF, FMP or any combination thereof, as compared to the general population of animals of that species, if the animal possesses: (i) a genetic marker genotype in linkage disequilibrium with a TT genotype in SCD g.23881050T>C; (ii) a genetic marker genotype in linkage disequilibrium with a GG genotype in FASN g.12323864A>G; and/or (iii) a genetic marker genotype in linkage disequilibrium with a AA genotype in FABP4 g.62829478A>T. Similarly, a genetic marker genotype in linkage disequilibrium with a TT genotype in SCD g.23881050T>C, a genetic marker genotype in linkage disequilibrium with a GG genotype in FASN g.12323864A>G, and/or a genetic marker genotype in linkage disequilibrium with a AA genotype in FABP4 g.62829478A>T, may be used to determine if a sheep or lamb is a ‘true to type’ Australian White lamb, in particular a ‘true to type’ Tattykeel Australian White (TAW) sheep/lamb.

In each of the foregoing examples, a genetic marker or marker genotype which is in LD with (i) SCD g.23881050T>C or a TT genotype in SCD g.23881050T>C, (ii) FASN g.12323864A>G or a GG genotype in FASN g.12323864A>G, or (iii) FABP4 g.62829478A>T or a AA genotype in FABP4 g.62829478A>T, will have an r² value of at least about 0.60 (e.g. about 0.60, or 0.65, or 0.70, or 0.75, or 0.80, or 0.85 or more). In one example, a genetic marker or marker genotype which is in LD with (i) SCD g.23881050T>C or a TT genotype in SCD g.23881050T>C, (ii) FASN g.12323864A>G or a GG genotype in FASN g.12323864A>G, or (iii) FABP4 g.62829478A>T or a AA genotype in FABP4 g.62829478A>T, has an r² value of at least 0.7.

SNP data for use in the method of the disclosure may be generated by any means known in the art. Exemplary methods are described herein. In some examples, the method of the disclosure may comprise generating the SNP genotype data from a nucleic acid sample from the ovine animal. Any method known in the art for generating SNP genotype data from a nucleic acid sample is contemplated.

The nucleic acid samples to be interrogated in the method of the disclosure may be prepared from any suitable biological sample obtained from the ovine animal. Thus, in some examples the methods of the disclosure may involve first taking a biological sample comprising nucleic acids from the animal to be tested. For example, the biological sample can be solid tissue, fibre or a biological fluid. Suitable biological fluids include, but are not limited to, whole blood, serum, plasma, cerebrospinal fluid, lymph fluids, semen, and various external secretions of the respiratory, intestinal and genitourinary tracts, tears, saliva, milk, white blood cells, and the like, and biological fluids such as cell extracts, cell culture supernatants, fixed tissue specimens, and fixed cell specimens. Suitable solid tissue samples include, but are not limited to, tissue from the ear (including cartilage), muscle, hair bulb or skin, including tissue biopsy or autopsy samples. A suitable fibre sample from which nucleic acid may be obtained includes wool. In some examples, the wool fibre may have a wool follicle or bulb attached which may also be a source of nucleic acids.

In some examples, the nucleic acid sample is prepared from tissue (e.g., muscle) taken from the longissimus dorsi thoracis et lumborum of the animal. In other examples, the nucleic acid sample is prepared from reproductive material of an animal selected from semen, an oocyte or an embryo. In other examples, the nucleic acid sample is prepared from blood or a blood fraction obtained from an animal. In other examples, the nucleic acid sample is prepared from a fibre (e.g., wool fibre and/or follicle attached thereto) obtained from an animal.

As used herein, the term “nucleic acid” or “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule can be single-stranded or double-stranded, but advantageously is double-stranded DNA. “DNA” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). An “isolated” nucleic acid molecule is one that is separated from other nucleic acid molecules that are present in the natural source of the nucleic acid e.g., a biological sample. In some examples, genomic DNA is preferred because the majority of SNPs within the genome are located in non-translated regions. However, for the avoidance of doubt, and where the context permits it, the term “nucleic acid sample” as used herein encompasses genomic DNA, cDNA, and mRNA.

The term “nucleoside” refers to a base linked to a sugar. The base may be adenine (A), guanine (G) (or its substitute, inosine (I)), cytosine (C), or thymine (T) (or its substitute, uracil (U)). The sugar may be ribose (the sugar of a natural nucleotide in RNA) or 2-deoxyribose (the sugar of a natural nucleotide in DNA). A “nucleotide” refers to a nucleoside linked to a single phosphate group.

The methods of the disclosure may comprise isolating the nucleic acid (e.g., DNA) from the biological sample prior to the step of determining the presence or absence of one or more of SNPs as described herein. The term “isolation”, “isolating” or “isolate” as used herein in the context of DNA, and as applied to the methods and assays described herein for preparing DNA from a sample, refers to and encompasses any method or approach known in the art whereby DNA can be obtained, procured, prepared, purified or isolated such that it is suitable for analysis, amplification and/or sequencing as provided in the methods and assays of the present disclosure. Various methods for the isolation or procurement of nucleic acid may be employed, as any skilled artisan may know and practice. Such methods may include methods employed for the isolation of DNA in various forms and states of purity and may not necessarily involve or require the separation of DNA from all cellular debris, protein, etc.

Whilst it is contemplated that SNP data for an animal may be available prior to performance of the method, in some examples the method may further comprise generating the SNP genotype data from a nucleic acid sample from the ovine animal. Any method known in the art for generating SNP genotype data from a nucleic acid sample is contemplated. Such methods include, but are not limited to, DNA sequencing (including amplicon sequencing), fluorescence spectroscopy, fluorescence resonance energy transfer (or “FRET”)-based hybridization analysis, high throughput screening, mass spectroscopy, selective nucleic acid hybridization, DNA amplification (including polymerase chain reaction (PCR)-based amplification methods), RFLP analysis and size chromatography (e.g., capillary or gel chromatography), and denaturing high-performance liquid chromatography (DHPLC), all of which are well known to one of skill in the art.

In some examples, the presence or absence of a SNP genotype described herein may be determined by sequencing the region of the genomic DNA sample that spans the polymorphic locus. For example, a DNA fragment spanning the location of the SNP of interest may be amplified using a suitable DNA amplification reaction. “Amplification” or “DNA amplification” as used herein refers to the increase in the number of copies of a particular nucleic acid target of interest, wherein the copies are also called “amplicons” or “amplification products”. Accordingly, the term “amplification assay”, as used in the context of DNA or nucleic acid amplification, shall be understood to refer to an assay configured to increase in the number of copies of a particular nucleic acid or DNA target of interest. Exemplary amplification assays are described herein. Illustrative methods include the polymerase chain reaction (PCR), long range PCR, amplification-refractory mutation system PCR (ARMS PCR), loop-mediated isothermal amplification (LAMP), rolling circle amplification (RCA), helicase dependant amplification (HDA), Nucleic Acid Sequence Based Amplification (NASBA), ramification amplification method (RAM), strand displacement amplification (SDA). recombinase-polymerase amplification (RPA), multiple strand displacement amplification (MDA), single primer isothermal amplification (SPIA), transcription mediated amplification (TMA), nicking enzyme amplification reaction (NEAR), exponential amplification reaction (EXPAR), smart-amplification process (SMAP) and others.

The amplification products or “amplicons” may then be analyzed in order to detect the presence or absence of specific SNPs (e.g., SCD g.23881050T>C, FASN g.12323864A>G or FABP4 g.62829478A>T). Detection and analysis of a SNP within a DNA amplicon or sequence can be performed using any method known in the art. Exemplary methods include the SNPStream® UHT Genotyping System (Beckman/Coulter, Fullerton, CA) (Boyce-Jacino and Goelet Patents), the Mass Array™ system (Sequenom, San Diego, CA) (Storm, N. et al. (2002) Methods in Molecular Biology. 2 2: 241-262.), the BeadArray™ SNP genotyping system available from Illumina (San Diego, CA)(Oliphant, A., et al. (June 2002) (supplement to Biotechniques), and TaqMan™ (Applied Biosystems, Foster City, CA). Additionally, the occurrence of particular nucleotides at SNP locations can be determined using a DNAMassARRAY system (SEQUENOM, San Diego, CA). This system combines proprietary SpectroChips™, microfluidics, nanodispensing, biochemistry, and MALDI-TOF MS (matrix-assisted laser desorption ionization time of flight mass spectrometry).

In other examples, detection and analysis of a SNP within a DNA amplicon or sequence can be performed by DNA sequencing. Any method or platform known in the art for sequencing DNA is contemplated for use in the method of the disclosure. Exemplary sequencing techniques, platforms and technologies, include, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, next-generation sequencing (NGS) and the like.

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variant′> (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyro-sequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3):333-43; and Zhang et al., “The impact of next-generation sequencing on genomics,” J Genet Genomics, 201, 38(3): 95-109.

In other example, SNP detection and analyses can also be performed via hybridization-based methods that comprise dynamic allele-specific hybridization (DASH) genotyping, using molecular beacons that employ a specifically engineered single-stranded oligonucleotide probe, SNP microarrays and the like. A broad range of enzymes including DNA ligase, DNA polymerase and nucleases have also been employed to generate high-fidelity SNP genotyping methods. These comprise restriction fragment length polymorphism (RFLP), PCR-based methods such as tetra-primer amplification refractory mutation system PCR (ARMS-PCR), oligonucleotide ligation assays and the like.

An ovine animal which is identified as having a desirable meat-eating quality trait, including n-3 LC-PUFA, IMF and FMP, following performance of the method of the disclosure, may be selected (e.g., from a population) for further animal breeding activities. Similarly, an ovine animal which is identified as being a ‘true to type’ Australian White sheep/lamb, in particular a Tattykeel Australian White Lamb (TAWL), may be selected (e.g., from a population) for further animal breeding activities. Methods of animal breeding, including production of reproductive materials for use in animal breeding, are described herein.

On the other hand, ovine animals which are identified as having genotypes at SNP locations SCD g.23881050T>C, FASN g.12323864A>G and/or FABP4 g.62829478A>T which are not associated with desirable meat-eating quality traits, may be selected (e.g., from a population) for removal from the population. Similarly, an ovine animal which is identified as not being a ‘true to type’ Australian White sheep/lamb may be selected (e.g., from a population) for removal from the population. Removal of an animal from a population may include physical segregation of the animal from the population, sale of the animal or culling of the animal.

### SNP Detection Systems

The present disclosure also provides a system for determining the nucleotide occurrence at one or more ovine SNPs selected from SCD g.23881050T>C, FASN g.12323864A>G and FABP4 g.62829478A>T. The system may comprise a hybridisation medium and/or substrate that comprises at least two oligonucleotides, each configured to hybridise selectively to a specific allele of an ovine SNP selected from SCD g.23881050T>C, FASN g.12323864A>G and FABP4 g.62829478A>T. For example, where the system is for determining the nucleotide occurrence at two or more (e.g., three) ovine SNPs selected from SCD g.23881050T>C, FASN g.12323864A>G and FABP4 g.62829478A>T, the hybridisation medium and/or substrate comprises at least two oligonucleotides for each SNP, wherein each oligonucleotide is configured to hybridise selectively to a specific allele of the respective ovine SNP. For example, a solid support may be provided, to which the oligonucleotides are directly or indirectly attached.

The oligonucleotides of the system are used to determine the nucleotide occurrence of ovine SNPs selected from SCD g.23881050T>C, FASN g.12323864A>G and FABP4 g.62829478A>T which the inventors have shown to be associated with meat-eating quality traits, such as IMF, FMP and n-3 LC-PUFA. The determination of the nucleotide occurrence of ovine SNPs can be made by selecting oligonucleotides that bind at or near a genomic location of the respective SNP(s) and which are designed to hybridise selectively to specific SNP allele. The system of the present disclosure typically includes a reagent handling mechanism that can be used to apply a reagent, typically a liquid, to the solid support. The ability of an oligonucleotide of the system to bind a nucleic acid from a genome of an ovine animal can be affected by the nucleotide occurrence at the SNP location. The system can include a mechanism effective for moving a solid support and a detection mechanism. The oligonucleotides of the system may be detectably labelled such that binding or tagging of the oligonucleotides may be detected (e.g., in a method of the disclosure). Detectable labeling of oligonucleotides is well known in the art and described herein. Particular non-limiting examples of detectable labels include chemiluminescent labels, fluorescent labels, radiolabels, enzymes, haptens, or even unique oligonucleotide sequences.

Detection and analysis of a SNP within a DNA amplicon or sequence using a SNP detection system of the disclosure can be performed using any method known in the art. Exemplary methods include the OvineSNP50 DNA Analysis Kit (Illumina, CA, USA), the Axiom™ Ovine Genotyping Array (Thermo Fisher, MA, USA), the Ovine 600K-SNP BeadChip (Illumina, CA, USA) and the Ovine Infinium HD 600K SNP BeadChip (Illumina, CA, USA)

### Test Kits

The present disclosure also provides a test kit which may be used in a method of the disclosure. That is, the test kit of the disclosure may be used to interrogate SNPs selected from SCD g.23881050T>C, FASN g.12323864A>G and/or FABP4 g.62829478A>T and thereby determine an animal's genotype at the respective SNP locations. This SNP genotype data can then be used to determine whether the ovine animal has or will have desirable FMP, IMF and/or n-3 LC-PUFA, and/or determine whether or not an ovine animal or part thereof is a ‘true to type’ Australian White Lamb or part thereof, in particular a Tattykeel Australian White Lamb (TAWL) or part thereof.

In one example, the present disclosure provides a test kit comprising a plurality of oligonucleotide primers and/or probes configured to discriminate between alleles at one or more SNP loci in biological samples taken from ovine animals, wherein the SNP loci are selected from (i) SCD g.23881050T>C or a SNP in linkage disequilibrium with SCD g.23881050T>C having an r² value of ≥0.7, (ii) FASN g.12323864A>G or a SNP in linkage disequilibrium with FASN g.12323864A>G having an r² value of ≥0.7, and/or FABP4 g.62829478A>T or a SNP in linkage disequilibrium with FABP4 g.62829478A>T having an r² value of ≥0.7.

As used herein, the term “primer” or similar refers to an enzymatically extendable oligonucleotide that comprises a defined sequence that is designed to selectively hybridize in an antiparallel manner with a complementary, primer-specific portion of a target (or template) nucleic acid sequence. This is referred to as “annealing” of the primer to its target. A primer which is generally provided in molar excess relative to its target polynucleotide sequence, primes template-dependent enzymatic DNA synthesis and amplification of the target sequence. Typically primers are used in performing polymerase chain reaction (PCR). A primer oligonucleotide sequence needn't have 100% complementarity with its template subsequence for primer elongation to occur. In this regard, primers with less than 100% complementarity can be sufficient for hybridization and enzymatic (e.g., polymerase) elongation to occur provided that there is sufficient complementarity for hybridisation to the target sequence and the penultimate base at the 3′ end of the primer is able to base pair with the template nucleic acid. A primer is preferably, but not necessarily, synthetic, and will generally be about 10 to about 100 nucleotides in length. Usually, however, primers contain about 15-26 contiguous nucleotides.

The term “probe” or “probes” refer to oligonucleotides sequences used in the detection of identical, similar, or complementary nucleic acid sequences by hybridization. An oligonucleotide can be of varying length but is typically of sufficient length to permit selective hybridisation, including to a specific target sequence.

An oligonucleotide sequence used as a primer or probe may be labelled with a fluorescent dye (or fluorophore) to facilitate detection of the amplification product. In some examples, the amplification product may be detectably labelled during the nucleic amplification process. For example, one or more of the oligonucleotide primers used in the amplification assay may be detectably labelled with a “fluorescent label” or “fluorescent dye” (also referred to as a “fluorophore”), such that the fluorescent dye is incorporated into the amplification product. As used herein, the term “fluorescent label” includes a signaling moiety that conveys information through the fluorescent absorption and/or emission properties of one or more molecules. Such fluorescent properties include fluorescence intensity, fluorescence life time, emission spectrum characteristics, energy transfer and the like.

Exemplary fluorescent dyes which may be conjugated or attached to a oligonucleotide primer include, but not limited to fluorescein/Oregon Green, fluorescein isothiocyanate (FITC), 6-Carboxyfluorescein, tetramethylrhodamine, Texas Red, dansyl, Alexa Fluor 488, BODIPY FL, lucifer yellow, and Alexa Fluor 405/Cascade Blue fluorophores. Fluorescent labels and their attachment to oligonucleotides are described in many reviews, including Haugland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); Wetmur (1991), Critical Reviews in Biochemistry and Molecular Biology, 26:227-259; and the like.

Commercially available fluorescent nucleotide analogues which may be readily incorporated into the polynucleotides during the nucleic acid amplification process include, for example, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHODAMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHODAMINE GREEN™-5-UTP, ALEXA FLUOR™ 488-5-UTP, ALEXA FLUOR™ 546-14-UTP (Molecular Probes, Inc. Eugene, Oreg.). Protocols are available for custom synthesis of nucleotides having other fluorophores. Henegariu et al., “Custom Fluorescent-Nucleotide Synthesis as an Alternative Method for Nucleic Acid Labeling,” Nature Biotechnol. 18:345-348 (2000).

Other fluorophores available for post-synthetic attachment include, inter alia, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, DYLIGHT™ DYES (e.g., DYLIGHT™ 405, DYLIGHT™ 488, DYLIGHT™ 549, DYLIGHT™ 594, DYLIGHT™ 633, DYLIGHT™ 649, DYLIGHT™ 680, DYLIGHT™ 750, DYLIGHT™ 800 and the like) (available from Thermo Fisher Scientific, Rockford, Ill.), Texas Red (available from Molecular Probes, Inc., Eugene, Oreg.), and Cy2, Cy3.5, Cy5.5, and Cy7 (available from Amersham Biosciences, Piscataway, N.J. USA, and others).

FRET tandem fluorophores may also be used, such as PerCP-Cy5.5, PE-Cy5, PE-Cy5.5, PE-Cy7, PE-Texas Red, and APC-Cy7; also, PE-Alexa dyes (610, 647, 680) and APC-Alexa dyes.

Metallic silver particles may be coated onto the surface of the array to enhance signal from fluorescently labelled oligonucleotide sequences bound to an array. Lakowicz et al. (2003) BioTechniques 34:62.

Labelling can also be carried out with quantum dots, as disclosed in the following patents and patent publications: U.S. Pat. Nos. 6,322,901; 6,576,291; 6,423,551; 6,251,303; 6,319,426; 6,426,513; 6,444,143; 5,990,479; 6,207,392; 2002/0045045; 2003/0017264; and the like.

The amplification products generated in a DNA amplification assay described herein may also be detected by a characteristic size, for example, on polyacrylamide or agarose gels stained with ethidium bromide. Alternatively, amplified target sequences may be detected by means of a detection probe (as described herein), which is an oligonucleotide tagged with a detectable label that hybridizes specifically to a target sequence in a nucleic acid, under conditions that allow hybridization, thereby allowing detection of the target sequence or amplification product. In one example, at least one tagged detection probe may be used for detection of allele-specific amplification product by hybridization. Preferably, the detection probe is designed to hybridize to a sequence in the target sequence that is between the amplification primers, i.e., it is an internal detection probe which hybridizes to the amplification product. The probe may be labelled to facilitate detection as described herein. Exemplary means of labelling oligonucleotides are described herein in context of the amplification primers and shall be taken to apply mutatis mutandis to each and every example describing a detection probe of the test kit.

Primers and probes are considered to be “selectively hybridizable” or “capable of selective hybridisation” if they are capable of specifically hybridizing to a nucleic acid template or a variant thereof (e.g., a specific SNP allele) or specifically prime a polymerase chain reaction under stringent conditions. Typical hybridization and wash conditions are described, for example, in Sambrook et al. supra and Nucleic Acid Hybridization, supra. Reduced stringency wash conditions that allow at most about 25-30% basepair mismatches include, for example: 2×SSC, 0.1% SDS, room temperature twice, 30 minutes each; then 2×SSC, 0.1% SDS, 37° C. once, 30 minutes; then 2×SSC room temperature twice, 10 minutes each. The selection of primers for use in typical polymerase chain reactions (PCR) under standard conditions is described for example, in Saiki, et al. (1988) Science 239:487-491. Preferably, primers and probes of the disclosure are capable of hybridizing under stringent conditions. That is, capable of selectively hybridizing to their cognate templates under stringent conditions.

The term “capable of hybridizing under stringent conditions” and the like as used herein refers to the ability of a primer and/or probe to hybridise or anneal to their cognate templates under stringent conditions. Stringent hybridization conditions typically permit the hybridization of nucleic acid molecules having at least 70% nucleic acid sequence identity with the nucleic acid molecule being used as a probe in the hybridization reaction. Hybridization of a probe or primer to its cognate template sequence may be conducted under stringent conditions, e.g., high temperature and/or low salt content that tend to disfavor hybridization of dissimilar nucleotide sequences. Alternatively, hybridization of a probe or primer to its cognate template sequence may be conducted under reduced stringency conditions, e.g. low temperature and/or high salt content that tend to favor hybridization of dissimilar nucleotide sequences. Low stringency hybridization conditions may be followed by high stringency conditions or intermediate medium stringency conditions to increase the selectivity of the binding of the primer and/or probe to the cognate templates. The hybridization conditions may further include reagents such as, but not limited to, dimethyl sulfoxide (DMSO) or formamide to disfavor still further the hybridization of dissimilar nucleotide sequences. A suitable hybridization protocol may, for example, involve hybridization in 6×SSC (wherein 1×SSC comprises 0.015 M sodium citrate and 0.15 M sodium chloride), at 65° C. in an aqueous solution, followed by washing with 1×SSC at 65° C. Formulae to calculate appropriate hybridization and wash conditions to achieve hybridization permitting 30% or less mismatch between two nucleic acid molecules are disclosed, for example, in Meinkoth et al. (1984) Anal. Biochem. 138: 267-284; the content of which is herein incorporated by reference in its entirety. Protocols for hybridization techniques are well known to those of skill in the art and standard molecular biology manuals may be consulted to select a suitable hybridization protocol without undue experimentation. See, for example, Sambrook et al. (2001) Molecular Cloning: A Laboratory Manual, 3rd ed., Cold Spring Harbor Press, the contents of which are herein incorporated by reference in their entirety.

Stringent conditions may be those in which the salt concentration is less than about 1.5 M sodium ion, typically about 0.01 to 1.0 M Na ion concentration (or other salts) from about pH 7.0 to about pH 8.3 and the temperature is at least about 30° C. for short probes (e.g., 10 to 50 nucleotide) an at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1-2×SSC at 50 to 55° C. Exemplary moderate stringency conditions include hybridization in 40 to 45% formamide, 1 M NaCl, 1% SDS at 37° C., and a wash in 0.5-1×SSC at 55 to 60° C. Exemplary high stringency conditions include hybridization in 50% formamide, 1 M NaCl, 1% SDS at 37° C., and a wash in 0.1×SSC at 60 to 65° C.

In one example, the test kit comprises:
one or more oligonucleotide primers and/or probes configured to discriminate between allele C and allele T at SNP locus SCD g.23881050T>C;
one or more oligonucleotide primers and/or probes configured to discriminate between allele G and allele G at SNP locus FASN g.12323864A>G; and/or
one or more oligonucleotide primers and/or probes configured to discriminate between allele A and allele T at SNP locus FABP4 g.62829478A>T.

In one example, the test kit may comprise one or more oligonucleotide primers and/or probes configured to discriminate between allele C and allele T at SNP locus SCD g.23881050T>C. In accordance with this example, the test kit may further comprise one or more oligonucleotide primers and/or probes configured to discriminate between allele G and allele G at SNP locus FASN g.12323864A>G. Alternatively, or in addition, the test kit may further comprise one or more oligonucleotide primers and/or probes configured to discriminate between allele A and allele T at SNP locus FABP4 g.62829478A>T. In a particular example, the test kit comprise one or more oligonucleotide primers and/or probes configured to discriminate between allele C and allele T at SNP locus SCD g.23881050T>C, one or more oligonucleotide primers and/or probes configured to discriminate between allele G and allele G at SNP locus FASN g.12323864A>G, and one or more oligonucleotide primers and/or probes configured to discriminate between allele A and allele T at SNP locus FABP4 g.62829478A>T.

In one example, the oligonucleotide primers in the test kit are configured to discriminate between allele C and allele T at SNP locus SCD g.23881050T>C by amplification-refractory mutation system polymerase chain reaction (ARMS PCR). Exemplary oligonucleotide primers configured to discriminate between allele C and allele T at SNP locus SCD g.23881050T>C by ARMS PCR comprise:
a primer comprising the sequence set forth in SEQ ID NO: 1 or a sequence which is substantially identical thereto (designated “SCD g.23881050T>C_Forward inner”);
a primer comprising the sequence set forth in SEQ ID NO: 2 or a sequence which is substantially identical thereto (designated “SCD g.23881050T>C_Reverse inner”);
a primer comprising the sequence set forth in SEQ ID NO: 3 or a sequence which is substantially identical thereto (designated “SCD g.23881050T>C_Forward outer (a)”);
a primer comprising the sequence set forth in SEQ ID NO: 4 or a sequence which is substantially identical thereto (designated “SCD g.23881050T>C_Reverse outer (a)”);
a primer comprising the sequence set forth in SEQ ID NO: 5 or a sequence which is substantially identical thereto (designated “SCD g.23881050T>C_Forward outer (b)”); and
a primer comprising the sequence set forth in SEQ ID NO: 6 or a sequence which is substantially identical thereto (designated “SCD g.23881050T>C_Reverse outer (b)”). These primers are also collectively referred to as the “SCD g.23881050T>C primers”.

In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 85% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 90% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 95% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 96% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 97% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 98% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be at least 99% identical to a primer sequence set forth in SEQ ID NO:1, SEQ ID NO:2, SEQ ID NO:3, SEQ ID NO:4, SEQ ID NO:5 and SEQ ID NO:6 respectively. In one example, one or more of the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) may be 100% identical to a primer sequence set forth in SEQ ID NO:1, SEQ ID NO:2, SEQ ID NO:3, SEQ ID NO:4, SEQ ID NO:5 and SEQ ID NO:6 respectively. In a particular example, the SCD g.23881050T>C primers designated SCD g.23881050T>C_Forward inner, SCD g.23881050T>C_Reverse inner, SCD g.23881050T>C_Forward outer (a), SCD g.23881050T>C_Reverse outer (a), SCD g.23881050T>C_Forward outer (b) and SCD g.23881050T>C_Reverse outer (b) comprise the sequences set forth in SEQ ID NO:1, SEQ ID NO:2, SEQ ID NO:3, SEQ ID NO:4, SEQ ID NO:5 and SEQ ID NO:6 respectively.

The final concentration of each of the SCD g.23881050T>C primers in the test kit may vary. In one example, the respective primers may each be present at a final concentration in the range of about 0.1 μM to about 2 μM. In one example, each of the primers is present at the same final concentration. In another example, one or more of the primers are present in the test kit at differing final concentrations (relative to the other primers in the test kit). For example, the outer primers and inner primers may be provided in a concentration ratio of about 2:1.

In accordance with some examples, two or more of the SCD g.23881050T>C primers are packaged together as a mixture. In other examples, the SCD g.23881050T>C primers are packaged separately.

The test kit may also comprise one or more or each of the following:
at least one DNA polymerase enzyme;
dinucleotide triphosphates (dNTPs);
a magnesium salt; and
a buffer.

A skilled person will appreciate that the choice of DNA polymerase will depend on the type of DNA amplification assay being employed. A skilled person would be able to select an appropriate enzyme based on the choice of amplification assay. For example, in accordance with an example in which the test kit is configured for a PCR amplification assay which relies on thermal conditions, a thermostable DNA polymerase (i.e., a DNA polymerase that originates from a thermophile) may be included in the test kit. A number of different thermostable DNA polymerases will be known to the person skilled in the art and are contemplated herein. The DNA polymerase enzyme is preferably a thermophilic DNA polymerase enzyme with strong strand displacement activity. Non-limiting examples of thermostable DNA polymerases suitable for inclusion in the test kit of the disclosure include naturally-occurring type A DNA polymerases (also known as family A DNA polymerases). Type A DNA polymerases are classified based on amino acid sequence homology to E. coli polymerase I (Braithwaite and Ito, (1993) Nuc. Acids. Res. 21:787-802), and include E. coli pol I, Thermus aquaticus DNA pol I (Taq polymerase), Thermus flavus DNA pol I, Streptococcus pneumoniae DNA pol I, Bacillus stearothermophilus pol I, phage polymerase T5, phage polymerase T7, mitochondrial DNA polymerase pol gamma, as well as additional polymerases discussed below. Family A DNA polymerases are commercially available, including Taq polymerase (New England BioLabs), E. coli pol I (New England BioLabs), E. coli pol I Klenow fragment (New England BioLabs), and T7 DNA polymerase (New England BioLabs), and Bacillus stearothermophilus (Bst) DNA polymerase (New England BioLabs).

In accordance with a different example in which the test kit is configured for an isothermal PCR amplification assay, a mesophilic enzymes for amplifying nucleic acids under isothermal conditions may be included in the test kit. The choice of mesophilic enzyme will depend on the choice of isothermal amplification assay used. A skilled person would be able to select an appropriate enzyme based on the choice of isothermal amplification assay. For example, where the test kit is configured for a LAMP assay, the kit will comprise one or more DNA polymerase enzymes suitable for use in a LAMP assay. The DNA polymerase enzyme is preferably a thermophilic DNA polymerase enzyme with strong strand displacement activity. For example, the DNA polymerase enzyme may be selected from the group consisting of Bst DNA polymerase, Bsm DNA polymerase, Gst DNA polymerase, SD DNA polymerase and combinations thereof.

The skilled person will understand that the amount of the DNA polymerase enzyme included in the test kit will depend on the number of reactions to be performed using the test kit. In this regard, the test kit may be scaled to accommodate any number of reactions. However, in one particular example, the test kit may comprise about 8 units of DNA polymerase per reaction. However, the skilled person will appreciate that this may be varied depending on the efficiency of the polymerase enzyme and particular DNA amplification assay employed.

As described herein, the reagent mixture will comprise dNTPs, which are the building block for synthesis of new DNA during the DNA amplification assay. The dNTPs will comprise dATP, dCTP, dGTP, and dTTP. These four dNTPs will preferably be present in equimolar amounts for optimal base incorporation during the DNA amplification assay. This is typically at a final concentration of about 0.2 mM for each dNTP. However, the relative molar amounts and the final concentrations may be varied as required. For example, higher concentrations of dNTPs may be desired in the presence of high levels of Mg²⁺, since Mg²⁺ binds to dNTPs and reduces their availability for incorporation into the new DNA strand during amplification. However, a skilled person will also appreciate that a concentration of dNTPs which is too high can also inhibit amplification. For example, a DNA amplification assay performed using the test kit may comprise dNTPs at a final concentration of about 0.8 mM to about 2 mM. In some examples, the test kit may be configured such that each assay comprises dNTPs within this concentration range.

The test kit may also comprise a magnesium salt. In this regard, magnesium ion (Mg²⁺) functions as a cofactor for activity of DNA polymerase by enabling incorporation of dNTPs during polymerization. The magnesium ions at the enzyme's active site catalyze phosphodiester bond formation between the 3′-OH of a primer and the phosphate group of a dNTP. In addition, Mg²⁺ facilitates formation of the complex between the primers or probe and the DNA template by stabilising negative charges on their phosphate backbones. The magnesium salt may be provided in any suitable form known to a person skilled in the art. For example, the magnesium salt may be magnesium chloride (MgCl₂) or magnesium sulphate (MgSO₄). In one example, the magnesium salt in the reagent mixture is MgSO₄. The concentration of magnesium salt may be optimised according to the conditions of the isothermal amplification assay e.g., by titration. In this regard, too low a Mg²⁺ concentration may result in little or no amplification product, due to the DNA polymerase's reduced activity. On the other hand, too high a Mg²⁺ concentration may result in non-specific amplification products as a result of enhanced stability of primer-template complexes, as well as increases in replication errors from misincorporation of dNTPs. A typical final concentration for Mg²⁺ in a DNA amplification assay is in the range of 1-10 mM. Accordingly, the test kit may be configured such that each assay comprises Mg²⁺ within this concentration range.

As described herein, the reagent mixture may also comprise a buffer to ensure a suitable chemical environment for activity of the DNA polymerase during the DNA amplification assay. There are a number of buffers known in the art and commercially available for use in nucleic acid amplification assays, and these are contemplated herein. The choice of buffer may depend on the choice of enzyme used for amplification (e.g., the choice of DNA polymerase). For example, where a Bst DNA polymerase is present in the reagent mixture (e.g., Bst 2.0 Warmstart® DNA polymerase), an amplification buffer suitable for Bst DNA polymerase (e.g., 10X Isothermal Amplification Buffer (Bst 2.0)) may be included in the reagent mixture.

In some examples, the test kit may be capable of being stored at freezing temperatures e.g., −20° C. or below.

In other examples, it is contemplated that certain component of the test kit (e.g., primers and/or probes) may be provided in a dried form e.g., lyophilised. This may assist with storage and transport at ambient temperatures. In accordance with an example in which certain reagents within the test kit are lyophilised, those reagents may further comprise a cryoprotectant. Suitable cryoprotectants for use in lyophilisation are known in the art and contemplated herein. The cryoprotectant may be a sugar selected from the group consisting of sucrose, trehalose, glucose, galactose, maltose, mannitol, lactose and derivatives thereof. The cryoprotectant may be present in an amount of about 7% w/v to about 8% w/v.

The test kit may also comprise one or more assay positive controls.

As described above, the test kit components may be packaged together as separate components. The test kit of the disclosure may further comprise instruction for use. The instructions for use may provide directions for using the test kit to determine an animal's genotype at the respective SNP locations in accordance with a method of one or more embodiments of the present disclosure. This SNP genotype data can then be used to determine whether the ovine animal has or will have desirable FMP, IMF and/or n-3 LC-PUFA, and/or determine whether or not an ovine animal or part thereof is a ‘true to type’ Australian White Lamb or part thereof, in particular a Tattykeel Australian White Lamb (TAWL) or part thereof

### Animal Breeding and Reproductive Materials

As described herein, an ovine animal which is identified as having a desirable meat-eating quality trait, including n-3 LC-PUFA, IMF and FMP, following performance of the method of the disclosure, may be selected (e.g., from a population) for animal breeding activities. Similarly, an ovine animal which is identified as being a ‘true to type’ Australian White sheep/lamb, in particular a Tattykeel Australian White Lamb (TAWL), may be selected for animal breeding activities.

Accordingly, in one example, the present disclosure provides a method of breeding an ovine animal which has been identified as having desirable n-3 LC-PUFA, IMF, FMP or any combination thereof according to the method of the disclosure. In other examples, the method of breeding an ovine animal of the disclosure comprises:
(i) selecting one or more animals having desirable n-3 LC-PUFA, IMF, FMP or any combination thereof, as compared to the general population of animals of that species based on the outcome of a method described herein which identifies one or more ovine animals having desirable n-3 LC-PUFA, IMF, FMP or any combination thereof, as compared to the general population of animals of that species; and
(ii) breeding from the selected animals to produce one or more offspring therefrom. In some examples, the method of breeding the animal comprises the step of performing the method as described herein for identifying one or more ovine animals having desirable n-3 LC-PUFA, IMF, FMP or any combination thereof, as compared to the general population of animals of that species. That is, prior to performing the selecting step.

As described herein, an animal having desirable n-3 LC-PUFA, IMF, FMP or any combination thereof, will comprise one or more SNP genotypes selected from a TT genotype in SCD g.23881050T>C, a GG genotype in FASN g.12323864A>G and/or an AA genotype in FABP4 g.62829478A>T. In this regard, the inventors have shown that (i) a TT genotype in SCD g.23881050T>C is associated with desirable IMF and n-3 LC-PUFAs as compared to a TA or TT genotype in SCD g.23881050T>C, (ii) a GG genotype in FASN g.12323864A>G is associated with desirable FMP, long chain saturated fatty acids and monounsaturated fatty acids, as compared to a GA or AA genotype in FASN g.12323864A>G, and (iii) an AA genotype in FABP4 g.62829478A>T is associated with IMF as compared to a AG or GG genotype in FABP4 g.62829478A>T. Thus, an animal bred to have desirable n-3 LC-PUFA, IMF, FMP or any combination thereof will comprise one or more of the aforementioned SNP genotypes.

A method of breeding an Australian White Lamb (AWL) is also provided, comprising:
(i) selecting one or more animals identified as having a SNP genotype indicative of AWL genetics; and
(ii) breeding from the selected animals to produce one or more offspring therefrom. In some examples, the method of breeding an AWL further comprises the step of performing the method as described herein on one or more animals to thereby identify one or more ovine animals having a SNP genotype indicative of AWL genetics. That is, prior to performing the selecting step.

A SNP genotype indicative of AWL genetics comprises (i) a TT genotype in SCD g.23881050T>C or a genotype in linkage disequilibrium with said TT genotype, (ii) a GG genotype in FASN g.12323864A>G or a genotype in linkage disequilibrium with said GG genotype, and/or (iii) a AA genotype in FABP4 g.62829478A>T or a genotype in linkage disequilibrium with said AA genotype. In some examples, the SNP genotype indicative of AWL genetics comprises one or more of a TT genotype in SCD g.23881050T>C, a GG genotype in FASN g.12323864A>G or a AA genotype in FABP4 g.62829478A>T. In one particular example, the SNP genotype indicative of AWL genetics comprises a TT genotype in SCD g.23881050T>C.

In accordance with the foregoing examples, the present disclosure also provides an ovine animal bred according to the method described herein.

Animal breeding can be performed by any means known to the skilled person. For example, breeding may be performed via natural service of a dam by a sire or using artificial or assisted reproductive technologies (ART). Artificial reproductive technologies may include artificial insemination, in vitro fertilisation (IVF), embryo transfer (ET), multiple ovulation embryo transfer (MOET), Juvenile in vitro embryo transfer (JIVET) or a combination thereof. In some examples, both the sire and the dam (or their respective reproductive materials) have been selected using a method of the disclosure. That is, both the sire and the dam (or their respective reproductive materials) have been selected on the basis that they have desirable meat-eating quality traits, including n-3 LC-PUFA, IMF and FMP, and/or are ‘true to type’ Australian White sheep/lamb, in particular a Tattykeel Australian White Lamb (TAWL). In other examples, just one of the sire and the dam (or the respective reproductive materials therefrom) have been selected on the basis that they have desirable meat-eating quality traits, including n-3 LC-PUFA, IMF and FMP, and/or are ‘true to type’ Australian White sheep/lamb, in particular a Tattykeel Australian White Lamb (TAWL). In the case of the latter example, it may be desired to introgress one or more of the SNP genotypes associated with desirable meat-eating quality traits, including n-3 LC-PUFA, IMF and FMP, into a population of ovine animals in which the SNP genotypes associated with the desirable meat-eating quality traits are absent or present in a lower than desirable frequency.

Reproductive or regenerative materials may also be obtained from an animal identified as having desirable n-3 LC-PUFA, IMF, FMP or any combination thereof according to the method of the disclosure, and/or which has been identified as being a ‘true to type’ Australian White sheep/lamb, in particular a Tattykeel Australian White Lamb (TAWL), according to the method of the disclosure.

“Reproductive material” as described herein shall include an oocyte, an embryo and/or semen. Similarly, “regenerative material” as used herein may be an oocyte, an embryo and/or semen, but may further includes primordial germ cells (PGCs) that give rise to oocytes or spermatozoa. Accordingly, the present disclosure contemplates the preparation of such reproductive or regenerative materials from animals selected using the method of the disclosure for future breeding activities. The reproductive or regenerative materials may be used fresh or cryopreserved and banked.

### EXAMPLES

### Example 1: Identification of SNP DNA Markers Through NGS to Select for Intramuscular Fat, Fat Melting Point Omega-3 Long Chain Polyunsaturated Fatty Acids and Meat Eating Quality in TattyKeel Australian White (TAW) MARGRA Lamb

The inventors utilized targeted NGS of functional SNPs in several lipid metabolism genes such as stearoyl-CoA desaturase (SCD), fatty acid binding protein-4 (FABP4), and fatty acid synthase (FASN) to identify unique DNA marker signatures for TAW genetics, breeding, and selection programs for meat-eating quality. This was performed on muscle tissue obtained from live lambs, using a minimally invasive longissimus dorsi thoracis et lumborum biopsy sampling technique, for the selection of omega-3 long-chain polyunsaturated fatty acids (n-3 LC-PUFA), intramuscular fat (IMF), and fat melting point (FMP) traits.

### Materials and Methods

### Animals and Experimental Design

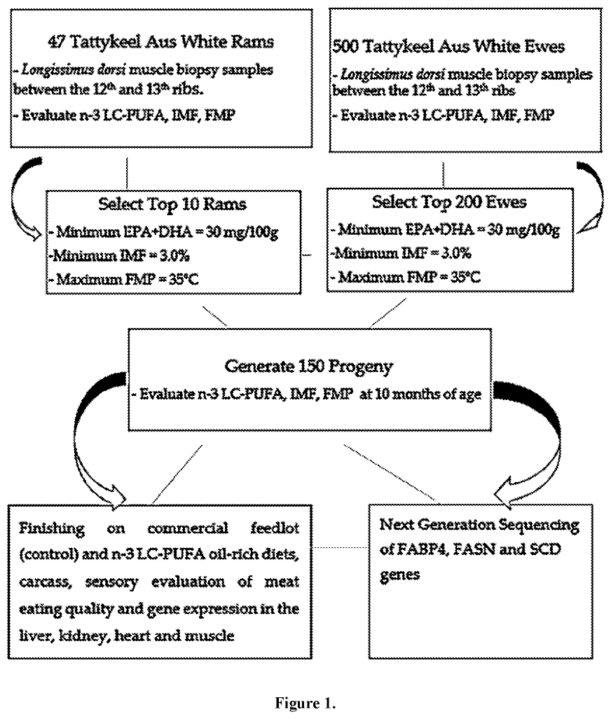

The experimental design for the selection, breeding, and evaluation of n-3 LC-PUFA, IMF, and FMP in Tattykeel Australian White (TAW) sheep is shown in FIG. 1.

Three composite generations—parental, first (F1), and second (F2) composite generations of lambs were bred, raised, and maintained under the same management at the Tattykeel Australian White Stud in Black Springs, Oberon, New South Wales, Australia. The parental composite generation comprised 47 rams mated to 500 ewes after evaluating their longissimus dorsi thoracis et lumborum muscle biopsy samples for health-beneficial n-3 LCPUFA, IMF, and FMP with minimum thresholds set at 30 mg/100 g, 3.0%, and 35° C., respectively. The top 10 rams and 200 ewes were selected and mated to generate 150 progeny whose muscle biopsy samples were laboratory tested for n-3 LC-PUFA, IMF, FMP, and genomic DNA sequenced at 10 months of age prior to being finished at a commercial feedlot. The Poll Dorset and Texel were used as positive control and the Rambouillet as the negative control in assessing extracted genomic DNA, polymerase chain reaction products, and next-generation sequencing procedures in the laboratory.

### Muscle Biopsy Sampling Procedure

The biopsy procedure for sampling the Longissimus dorsi muscle from the 12th-13^{th} ribs was first described in cattle [Malau-Aduli, A. E. O. et. al 1998. J. Anim. Sci. 76, 766-773] and modified in sheep [Pewan, S. B. et. al 2020. Antioxidants. 9, 1118]. This method has also been described in further detail in Pewan, S. B. et. al 2020. Antioxidants. 9, 1118.

### Determination of Intramuscular Fat

Details of the procedures for laboratory analysis of intramuscular fat have been described in further detail in Pewan, S. B. et. al 2020. Antioxidants. 9, 1118, Holman, B. et. al 2014. Int. J. Vet. Med. Res. Rep. 1-9, and Flakemore, A. R. et. al 2014. Int. J. Nutr. Food Sci. 3, 203-209.

### Determination of Fat Melting Point

Details of the laboratory analysis of fat melting point have been described in further detail in Pewan, S. B. et. al 2020. Antioxidants. 9, 1118, Holman, B. et. al 2014. Int. J. Vet. Med. Res. Rep. 1-9, and Flakemore, A. R. et. al 2014. Int. J. Nutr. Food Sci. 3, 203-209.

### Determination of Fatty Acid Composition

Fatty acid composition including n-3 LC-PUFA analysis of Longissimus dorsi muscle biopsy samples was analyzed by means of gas chromatography-mass spectrophotometry procedure described in detail by Malau-Aduli, A. E. O. et. al 2016. Anim. Prod. Sci. 56, 2122-2132 based on modified methods in Bligh, E. G. et. al 1959. Can. J. Biochem. Phys. 37, 911-917, Miller, M. R. et. al 2006. Lipids 2006, 41, 865-876 and Clayton, E. 2014. Graham Centre Monograph no. 4; Charles Sturt University: Wagga-Wagga, NSW, Australia. Details have been described in further detail in Pewan, S. B. et. al 2020. Antioxidants. 9, 1118.

### Blood Collection and Genomic DNA Extraction

Approximately 10 mL of blood was collected from Tattykeel Australian White, Poll Dorset, and Texel (positive control) lambs of the same age and under the same management conditions by jugular venipuncture into vacutainers containing EDTA. Blood samples were stored at −80° C. until ready for genomic DNA (gDNA) extraction. gDNA was extracted from 2 mL of blood using NucleoSpin Blood Kits (Macherey-Nagel GmbH and Co. KG, Neumann-Neander-Str. 6-8. 52355 Duren, Germany) according to the manufacturer's protocol. gDNA yield was quantified with a NanoDrop ND-1000 spectrophotometer (NanoDrop, Thermo Fisher Scientific Australia Pty Ltd, Scoresby, Victoria, Australia).

### Primer Design for FASN, FABP4 and SCD

All primers were designed using Geneious Prime Software Program 2020 v.2.2 (http://www.geneious.com). A targeted candidate gene approach of lipid metabolism genes (FASN, FABP4, and SCD) was utilized. Single coding sequences of each gene deposited in the National Center for Biotechnology Information (NCBI) database (Genbank) of FASN, FABP4, and SCD of Ovis aries breed were used as reference points. In order to amplify the 18 kb of the FASN gene (Accession Number: NC_040262.1), a long-range PCR approach was used to split the gene sequence into 3 overlapping fragments of 8.5 kb each (FASN1, FASN2, and FASN3), comprising approximately 91% of the total gene sequence. For the 4 kb FABP4 (NC_040260.1) and 12 kb SCD (NC_040273.1) gene fragments, a single primer set was designed as shown in Table 1. All primers were synthesized at Integrated DNA Technologies Pte. Ltd., Melbourne, Australia.

### Long-Range PCR

Due to the different fragment lengths and DNA composition, it was necessary to use 3 different long-range PCR approaches to amplify the FASN, FABP4, and SCD genes. During optimization, all 3 approaches were tested for all 3 genes, but only the best performing combinations were utilized.

### FASN Amplification

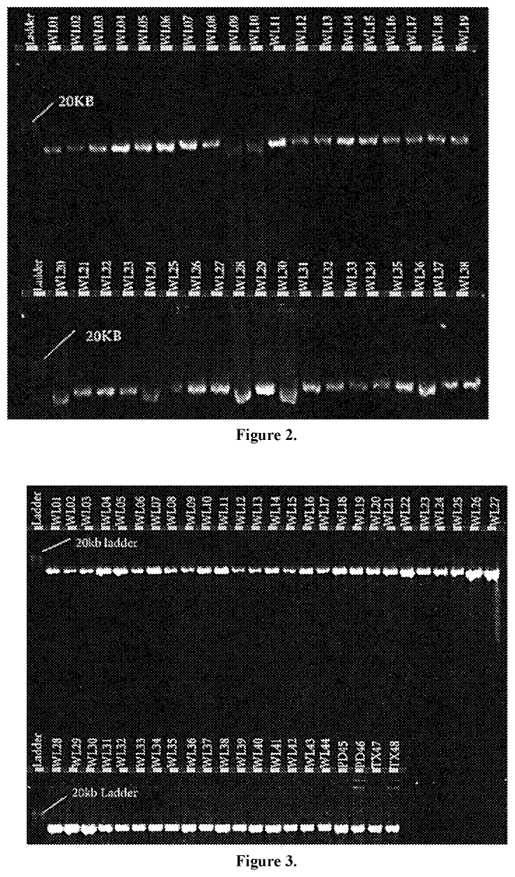

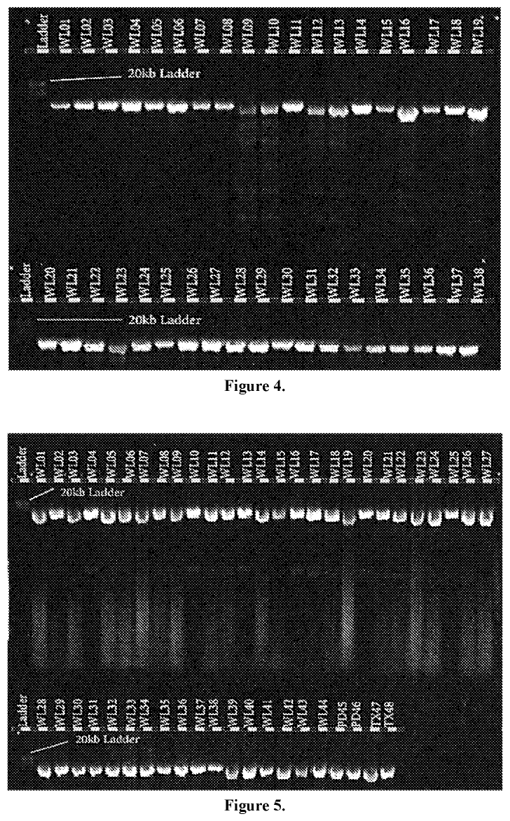

FASN PCR amplification assay was performed using the TakaRa PrimeSTAR GXL Master Mix (TaKaRa Bio Inc., Kusatsu, Shiga, Japan). PCR reaction assay was set up in a total volume of 50 μL containing 10 μL of 5× TakaRa PrimeSTAR GXL Buffer, 200 μM of TaKaRa dNTP Mixture, 1.25 units of TaKaRa PrimeSTAR GXL DNA Polymerase, 0.2 μM of each primer (IDT, Melbourne, Australia), and 100 ng of DNA template. PCR was performed in a SimpliAmp™ Thermal Cycler (Thermofisher Scientific, Melbourne, Australia), in a 2-step protocol using the following conditions: 98° C. initial denaturation for 1 min (1 cycle); 98° C. denaturation for 10 s; 68° C. annealing/extension for 10 min for 30 cycles. PCR success was checked in 0.8% agarose gel electrophoresis as depicted in FIGS. 2-4.

### FABP4 and SCD Amplification

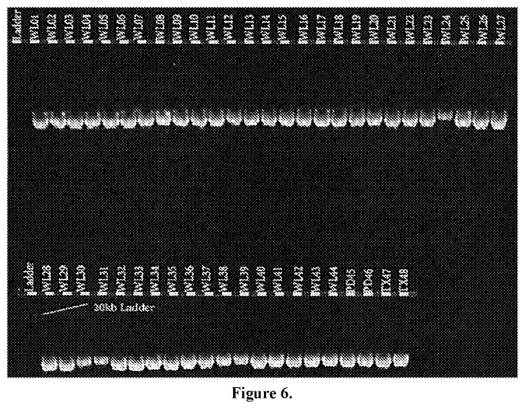

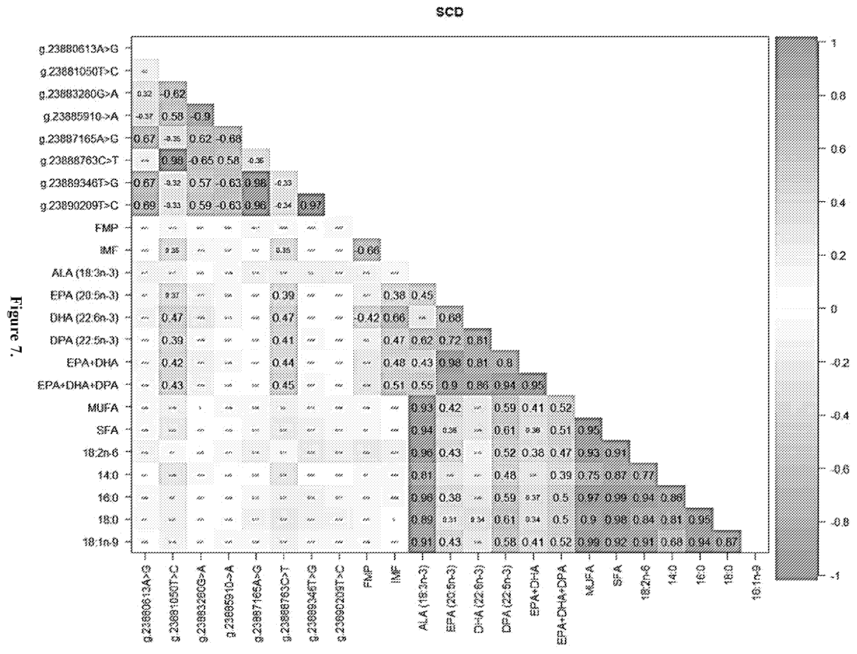

For the FAPB4 gene, Platinum™ SuperFi™ II PCR Master Mix (Thermofisher Scientific, Australia) was used, while for the SCD gene, Hot Start II High-Fidelity PCR Master Mix (Thermofisher Scientific, Australia) was used under the same PCR conditions. The amplification reactions were performed in a total volume of 50 μL containing 25 μL of 2×Platinum™ SuperFi™ II PCR Master Mix or Phusion Hot Start II High-Fidelity PCR Master Mix (Thermofisher Scientific, Australia), 0.5 μM of each primer (IDT, Australia), and 100 ng of DNA template. PCR was performed in a SimpliAmp™ Thermal Cycler (Thermofisher Scientific, Australia), in a 3-step protocol, using the following conditions: 98° C. initial denaturation in 1 min (1 cycle); 98° C. for denaturation 15 s; 60° C. (FABP4)/and 65° C. (SCD) annealing for 15 s; 72° C. extension for 9 min; 72° C. final extension for 9 min; 4° C. hold for 35 cycles. PCR success was checked in 0.8% agarose gel electrophoresis, as depicted in FIGS. 5 and 6.

### PCR Clean-Up

Sera-Mag™ SpeedBeads was prepared according to Faircloth, B. C. et. al 2014. Illumina Library Prep Protocol. Release 2.1 and used to clean the PCR products using a Zephyr NGS Workstation (Caliper Lifesciences, Perkin-Elmer) and quantified using a Promega dsDNA Quantifluor System Kit (Ref E2670, 00002484139) on an Enspire Workstation (Perkin-Elmer). The 5 different PCR products were pooled at approximately 0.4 nM to ensure even coverage during sequencing using Quantifluor dsDNA System (Promega, Madison, WI, USA). The products were normalized to 2 ng/μL using 10 mM Tris-HCl (pH 8.0). Final dilution to 0.2 ng/μL with 10 mM Tris-HCl (pH 8.0) was conducted in preparation for library preparation and final accuracy checks using the Illumina NexteraXT DNA.

### Library Preparation, Quantification, Normalization, and Sequencing

Libraries were prepared using Nextera XT DNA Library Prep kit (Illumina, Ca, USA) in accordance with the manufacturer's protocols using the recommended input of 5 μL of 0.2 ng/μL gDNA per sample. This was followed by Sera-Mag™ SpeedBeads purification using 0.6× beads and 2 washes using 80% ethanol to select fragments>250 bp and remove unincorporated adapters. Each DNA library fragment size and concentration was determined using Agilent High Sensitivity D5000 reagents and ScreenTape on the Tape Station 4200 Instrument (Agilent Technologies, Santa Clara, CA, USA) according to the Agilent assay quick guide. Additionally, all individual libraries were quantified using Quanti-Fluor® dsDNA System (Promega, Madison, WI, USA) to give an additional concentration estimate. The resultant size and concentration data from Tape Station and Quantifluor system were used to normalize each library to 4 nM by diluting with 10 mM Tris-HCl (pH8.5) prior to pooling. An equal volume of 5 μL was pooled and sequenced on an Illumina MiSeq benchtop sequencer, using a 500-cycle MiSeq Reagent Nano Kit v2 with a 10 pM input and 10% PhiX spike-in.

### Bioinformatics and Next Generation Sequencing Data Analysis

Genomic data analysis was performed using commercial bioinformatics program Geneious Prime software program 2020 v.2.24 (http://www.geneious.com (accessed on 12 Jun. 2021)) to analyze the sequences. The following reference sequences deposited in the NCBI database were used for comparative analysis: NC_040262.1, NC_040260.1, and NC_040273.1 for FASN, FABP4, and SCD genes, respectively. Next Generation Sequenced data were retrieved from Illumina Dashboard-BaseSpace Sequence Hub (https://basespace.illumina.com/dashboard (accessed on 15 Jul. 2021) as paired read data in 2 separate forward and reverse read lists in FASTQ format. The retrieved raw reads were subjected to quality control measures. Reads were trimmed and adapters removed using the BBDuk trimmer in Geneious Prime 2020 v.2.2 with the default setting for paired end reads. The Quality (Q) value of Phred score was set at 20 to improve sequenced data and increase the likelihood of calling true SNPs to 99%. Short reads with a minimum length of 20 bp were discarded, resulting in clean reads. Regions of low coverage were excluded when calling SNPs using the Annotate and Predict→Find Low/High Coverage. The reads were mapped to reference in Geneious. The reference sequences were retrieved from NCBI database (Genbank) of FASN, FABP4, and SCD of Ovis aries breed. The Sensitivity was set on the Medium Sensitivity/Fast and Fine-Tuning (iterate up to 5 times) option selected to improve the results by aligning reads to each other in addition to the reference sequence. Major allele frequencies from the next-generation sequence data based on observed and expected genotypes were computed using the Hardy-Weinberg equilibrium principle as described by Graffelman, J. et. al. 2017. Hum. Genet. 136, 727-741.

### Statistical Analyses

All statistical analyses of the associations between detected SNP of the 3 genes and meat-eating quality traits were performed using R statistical software version 3.6.3 (http://www.R-project.org/). Linkage disequilibrium as an index of non-random association between alleles of different loci, was estimated as the difference between the frequency of gametes carrying the pair of alleles A and B at two loci (pAB) and the product of the frequencies of those alleles (pA and pB), D_{AB}=pAB−pApB, where the allele pair AB is a haplotype and pAB is the haplotype frequency [Slatkin, M. 2008. Nat. Rev. Genet. 9, 477-485]. Major and minor allele frequencies were computed, and the Hardy-Weinberg Equilibrium was tested using the chi-square test. Pearson's residual correlation analysis was carried out to examine the relationships between genomic variants and meat quality traits (FA, FMP, and IMF). Linear mixed models procedure was used to investigate differences in FMP, IMF, and fatty acid profiles of the TAW lambs due to FABP4, SCD, and FASN variants fitting the fixed effect of allele substitution for individual SNP and random effect of animal (for pedigree) accounting for composite generation effects. Functional allele mutations at the coding regions of identified FABP4, SCD, and FASN loci were statistically analyzed for association with FMP, IMF, and fatty acids. Least-square means were compared using the Tukey-adjusted multiple comparisons test. The full statistical model was:

$${Y}_{ij} = μ + {α}_{i} {+}_{γ 1} {FA}_{ij} {+}_{γ 2} {SC}_{ij} + {+}_{γ 3} {SK}_{ij} + {e}_{ij}$$

where:
Yᵢⱼ=dependent variable (FMP, IMF, FA) of j^{th} TAW of i^{th} composite generation;
μ=overall mean;
αᵢ=effect of the i^{th} composite generation;
FA=the genotype FASN (AA, GA and GG);
SC=the genotype SCD (CC, CT and TT);
SK=the genotype FABP4 (GG, GA and AA);
γ=effect of the genotype; and
eᵢⱼ=residual error.

### Results

This study of SCD, FASN, and FABP4 lipogenic genes SNP in TAW lamb muscle biopsy samples bred, selected, and evaluated as per the experimental design shown in FIG. 1, was based on the Geneious-designed primers whose sequences are presented in Table 1 and successful polymerase chain reactions (PCR) products are presented in FIGS. 2-6.

TABLE 1
Primer sequences for FABP4, FASN, and SCD polymerase chain reaction assays^{#}.

| | | | Length | Tₐ | Fragment Length |
|---|---|---|---|---|---|
| Gene | | Sequence | (bp) | (° C.) | (bp) |
| FASN 1 | Forward | CCTACTTTCCCATGCTCAGAGAA | 23 | 68 | |
| | Reverse | CTACGTTGCTGAGGAAGAACTCTA | 24 | 68 | |
| FASN 2 | Forward | ACCGTCTCTCCTTCTTCTTTGAC | 23 | 68 | |
| | Reverse | GAAGTTGAGGGAGGCGTAATAGAT | 24 | 68 | |
| FASN 3 | Forward | CTAGAGTTCTTCCTCAGCAACGTA | 24 | 68 | |
| | Reverse | GCCAGGGACCTGTGAATAATACTA | 24 | 68 | |
| FABP4 | Forward | TTGTTGAATGGCTGGGCTTATAAC | 24 | 60 | 4107 |
| | Reverse | TAAGAAAATACTTCCTGGGGCACA | 24 | 60 | |
| SCD | Forward | CAAACTTAGGTCTGCAACTTTCGT | 24 | 65 | 11,545 |
| | Reverse | TTTCCCACTTCAACTCACCCTATT | 24 | 65 | |

^{#}FASN, Fatty Acid Synthase; FABP4, Fatty Acid Binding Protein 4; SCD, Stearoyl-CoA Desaturase T., annealing temperature.

### SCD, FASN, and FABP4 Gene SNP Variants and Genotypes

Using the Poll Dorset and Texel as positive controls, and Rambouillet as negative controls, eight SCD gene SNP loci (g.23880613A>G; g.23881050T>C; g.23883280G>A; g.23885910C>A; g.23887165A>G; g.23888763C>T; g.23889346T>G; g.23890209T>C) with major allele frequencies ranging from 0.53 to 0.93 were identified as depicted in Table 2. It was evident from Table 2 that TAW lambs were all heterozygous at three loci (g.23881050T>C, g.23883280G>A g.23885910C>A) in the parental, first, and second composite generations, thereby presenting a genetic divergence from the homozygous variants seen in the Poll Dorset, Texel and Rambouillet controls.

TABLE 2
SCD gene SNP (major allele frequency) in TAW 1, Poll Dorset (+ control), and Rambouillet (− control) lambs.
Lamb breed, generation, type of control and genotypes (major allele frequencies in brackets)
Parental composites 1^{st} ( [symbol] ) and 2^{nd} ( [symbol] ) composites Positive (+) and negative (−) controls

| | TAW Parents | | TAW [symbol] | | TAW [symbol] | | Poll Dorset | Texel | Rambouillet |
|---|---|---|---|---|---|---|---|---|---|
| SNP locus | (n = 147) | | (n = 75) | | (n = 75) | | (+ n = 2) | (+ n = 2) | (− n = 2) |
| g.23880613A > G | GG | (0.82) | GG | (0.93) | GG | (0.73) | GG | GG | AA |
| g.23881050T > C | CT | (0.58) | CT | (0.54) | CT | (0.90) | CC | CC | TT |
| g.23883280G > A | AG | (0.53) | AG | (0.71) | AG | (0.60) | AA | AA | GG |
| g.23885910C > A | AC | (0.57) | AC | (0.71) | AC | (0.53) | CC | CC | CC |
| g.23887165A > G | GA | (0.69) | GG | (0.82) | GG | (0.70) | GG | GG | AA |
| g.23888763C > T | TC | (0.58) | TC | (0.54) | CC | (0.93) | CC | CC | CC |
| g.23889346T > G | GT | (0.68) | GG | (0.82) | GG | (0.70) | GG | GG | TT |
| g.23890209T > C | CT | (0.67) | CC | (0.82) | CC | (0.70) | CC | CC | TT |

[symbol]

TAW, Tattykeel Australian White. [symbol]
indicates data missing or illegible when filed

As depicted in Table 3, nine functional SNP covering 91% of the FASN gene sequence were identified. The genotypes at the nine loci were all the same in TAW, indicating a consistent heredity pattern from the composite TAW parents to the first and second generations, which were all distinguishable from the Rambouillet negative control breed.

TABLE 3
FASN gene SNP (major allele frequency) in TAW 1, Poll Dorset (+ control) and Rambouillet (− control) lambs.
Lamb breed, generation, type of control, and genotypes (major allele frequencies in brackets)
Parental composites 1st [symbol] and 2nd [symbol] composites,Positive (+) and negative (−) controls

| | TAW Parents | | TAW [symbol] | | TAW [symbol] | | Poll Dorset | Texel | Rambouillet |
|---|---|---|---|---|---|---|---|---|---|
| SNP locus | (n = 147) | | (n = 75) | | (n = 75) | | (+ n = 2) | (+ n = 2) | (− n = 2) |
| g.12316077T > G | GG | (0.89) | GG | (0.86) | GG | (0.95) | GG | GG | TT |
| g.12318491A > G | GG | (0.89) | GG | (0.86) | GG | (0.95) | GG | GG | AA |
| g.12320583T > C | CC | (0.89) | CC | (0.86) | CC | (0.97) | CC | CC | TT |
| g.12321671T > C | CC | (0.89) | CC | (0.86) | CC | (0.97) | CC | CC | TT |
| g.12323864A > G | GA | (0.70) | GA | (0.69) | GA | (0.70) | GG | GG | AA |
| g.12324288G > A | AG | (0.69) | AG | (0.68) | AG | (0.69) | AA | AA | GG |
| g.12326992T > C | CC | (0.88) | CC | (0.79) | CC | (0.90) | CC | CC | TT |
| g.12327084 −> CT | CT | (0.50) | CT | (0.50) | CT | (0.50) | CT | CT | TT |
| g.12328120T > C | CC | (0.89) | CC | (0.86) | CC | (0.97) | CC | CC | TT |

[symbol]

TAW, Tattykeel Australian White. [symbol]
indicates data missing or illegible when filed

For the FABP4 gene, three SNP loci were genotyped with major allele frequencies ranging from 0.50 to 0.97 (Table 4).

TABLE 4
FABP4 gene SNP (major allele frequency) in TAW 1, Poll Dorset
(+ control) and Rambouillet (− control) lambs.
Lamb breed, generation, type of control, and genotypes
(major allele frequencies in brackets)
Parental composites 1st [symbol] and 2nd [symbol] composites
Positive (+) and negative (−) controls

| | TAW Parents | | TAW [symbol] | | TAW [symbol] | | Poll Dorset | Texel | Rambouillet |
|---|---|---|---|---|---|---|---|---|---|
| SNP locus | (n = 147) | | (n = 75) | | (n = 75) | | (+ n = 2) | (+ n = 2) | (− n = 2) |
| g.62826961T > C | CT | (0.61) | TT | (0.64) | CT | (0.60) | TT | TT | TT |
| g.62826965C > G | GC | (0.61) | GC | (0.57) | GC | (0.60) | GG | GG | CC |
| g.62829478A > T | AT | (0.55) | AT | (0.61) | AT | (0.53) | AA | AA | AA |

[symbol]

TAW, Tattykeel Australian White. [symbol]
indicates data missing or illegible when filed

### Correlations Between SCD, FASN, and FABP4 Gene SNP, FMP, IMF, and Fatty Acids

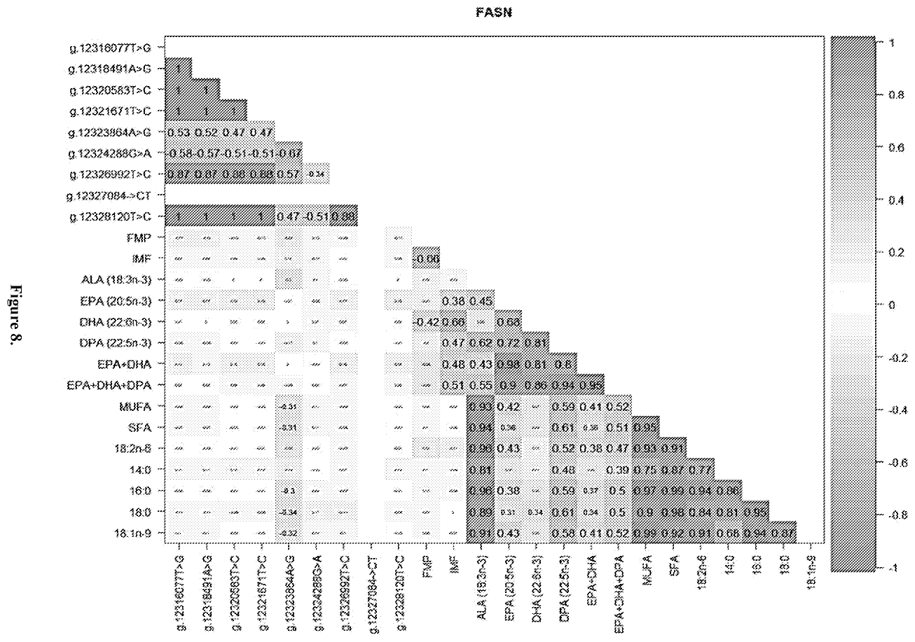

FIG. 7 shows significant correlations between detected SCD SNP loci, several fatty acids and other meat-eating quality traits. Among SCD SNP loci, the highest correlations of 0.98 were observed between g.23888763C>T and g.23881050T>C; g.23889346T>G and g.23887165A>G. Moderate correlations between health-promotingn-3 LC-PUFA (EPA, DHA, and DPA), and g.23888763C>T and g.23881050T>C loci ranging from 0.37 to 0.47 were observed. IMF was moderately to highly correlated with n-3 LC-PUFA (0.38-0.66), while FMP was negatively correlated with IMF (−0.66) and DHA (−0.42). Among the different fatty acids and their summations, very high correlations of up to 0.99 were evident (FIG. 7).

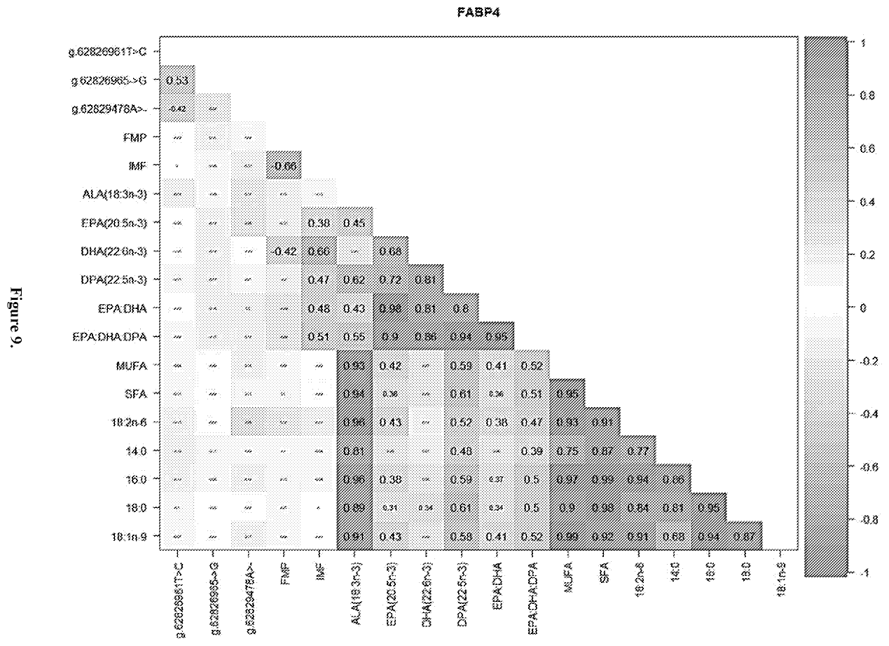

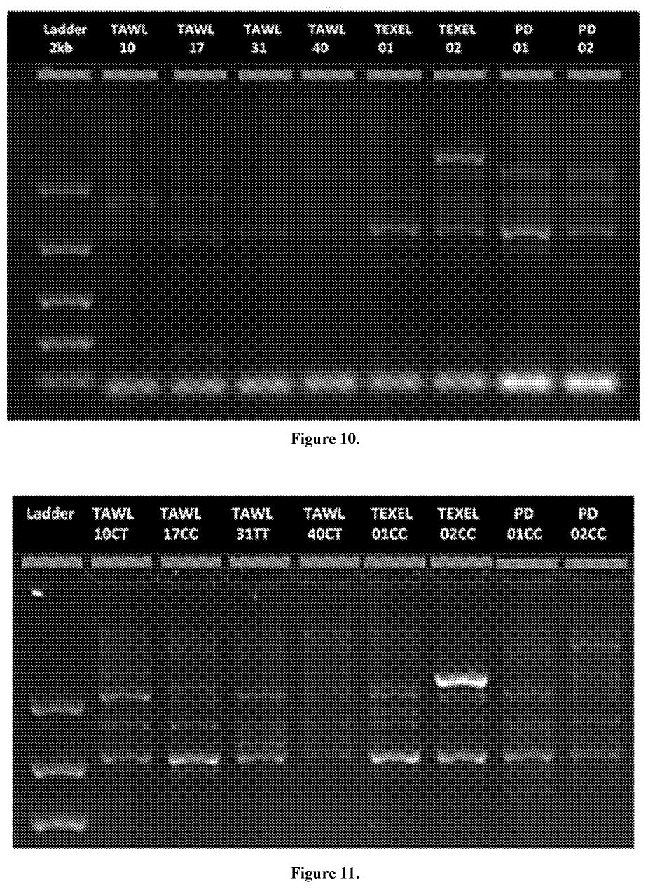

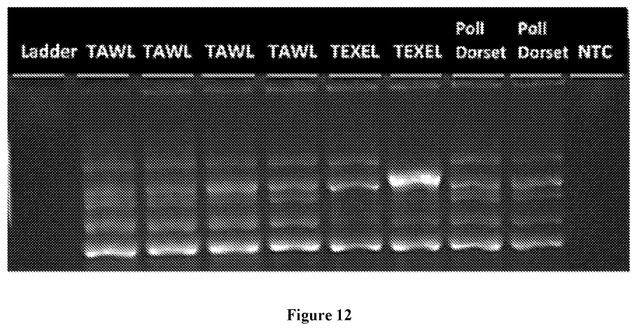

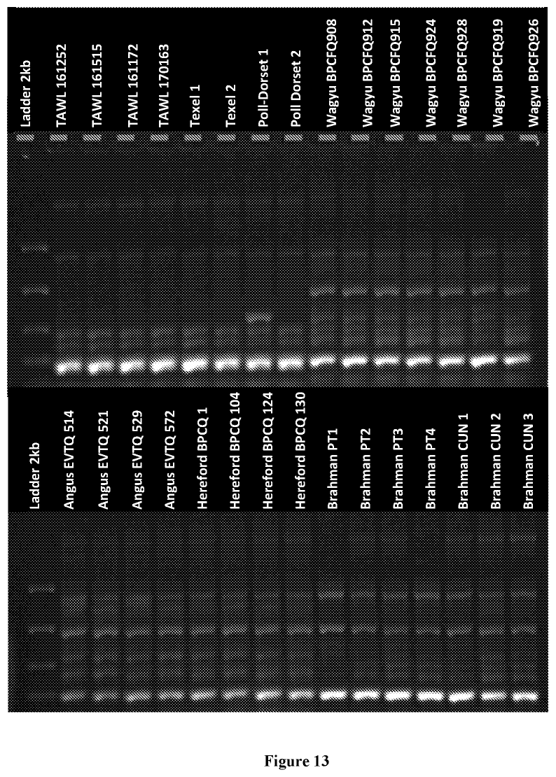

FIG. 8 shows that among FASN gene SNP, there were highly significant correlations between the loci, while correlations between the g.12323864A>G locus and most fatty acids were negative, ranging from −0.3 to −0.34. Negative correlations between IMF and FMP (−0.66) and DHA (−0.42) were also observed, while the highest positive correlations were between the various fatty acids (FIG. 8).

FIG. 9 shows that among FABP4 gene SNP, the highest correlation of 0.53 was between the loci g.62826965C>G and g.62826961T>C, while a negative correlation of −0.42 was observed between g.62826965C>G and g.62829478A>T. Consistently positive correlations between IMF and n-3 LC-PUFA of up to 0.66 with DHA, 0.47 with DPA, and 0.38 with EPA were also observed, while the highest positive correlations were among the various fatty acids and their summations (FIG. 9).

### Associations Between SCD, FASN and FABP4 SNP, FMP, IMF, and Fatty Acids

Descriptive statistics of mean, standard deviation, and coefficient of variation of the meat quality traits and full suite of fatty acids breakdown are presented in Table 5. FMP had a mean of 33.65° C. with a standard deviation of 2.74 and coefficient of variation of 8.14%, while IMF averaged 4.43% with a standard deviation of 1.31 and coefficient of variation of 29.58%. Table 5 also shows that the SCD g.23881050T>C SNP was significantly associated with IMF (p<0.0089) and DHA (p<0.0111), while FABP4 g.62829478A>G SNP was associated with only IMF (p<0.0539). The FASN g.12323864A>G SNP was associated with FMP (p<0.0544), ALA (p<0.0033), MUFA (p<0.0025), SFA (p<0.0025), C18:2n-6 (p<0.0138), C16:0 (p<0.0039), C18:0 (p<0.0012) and C18:1n-9 (p<0.0023) fatty acids (Table 5).

TABLE 5
Associations between SNP mutations and FMP, IMF, and fatty acids in TAW lambs^{#}.
Tukey-Adjusted Multiple Comparison Tests for Significant SNP, FMP, IMF, and Fatty Acids

| | | | SNP effect (p-values) | | | |
|---|---|---|---|---|---|---|
| | | | SCD | FABP4 | FASN | |
| Variable | Mean | SD | CV (%) | g.23881050T > C | g.62829478A > T | g.12323864A > G |
| FMP (° C.) | 33.65 | 2.74 | 8.14 | 0.2700 | 0.6115 | 0.0544 * |
| IMF (%) | 4.43 | 1.31 | 29.58 | 0.0089 ** | 0.0539 * | 0.1915 |
| Fatty acids (mg/100 g) | | | | | | |
| ALA (C18:3n-3) | 163.03 | 192.27 | 117.94 | 0.7755 | 0.1419 | 0.0033 ** |
| EPA (C20:5n-3) | 25.20 | 11.62 | 46.10 | 0.7683 | 0.1023 | 0.9810 |
| DHA (C22:6n-3) | 8.43 | 4.16 | 49.27 | 0.0111 * | 0.2145 | 0.9480 |
| DPA (C22:5n-3) | 23.85 | 13.70 | 57.44 | 0.0532 * | 0.3894 | 0.0927 |
| EPA + DHA | 33.64 | 14.75 | 43.84 | 0.2036 | 0.4794 | 0.9915 |
| EPA + DHA + DPA | 57.49 | 26.97 | 46.92 | 0.0728 | 0.8958 | 0.2004 |
| MUFA | 3694.70 | 4099.08 | 110.94 | 0.6824 | 0.3949 | 0.0025 ** |
| SFA | 4392.18 | 5238.81 | 119.28 | 0.4000 | 0.5472 | 0.0029 ** |
| C18:2n-6 | 253.68 | 247.70 | 97.64 | 0.6781 | 0.0647 | 0.0138 * |
| C14:0 | 287.92 | 437.58 | 151.98 | 0.0632 | 0.7354 | 0.1190 |
| C16:0 | 2076.17 | 2419.46 | 116.53 | 0.5414 | 0.3751 | 0.0039 ** |
| C18:0 | 1683.83 | 2065.71 | 122.68 | 0.3891 | 0.9125 | 0.0012 ** |
| C18:1n-9 | 2901.10 | 3212.65 | 110.74 | 0.8555 | 0.3696 | 0.0023 ** |

[symbol]

p < 0.05, [symbol]
p < 0.01;
SFA, Saturated fatty acids;
MUFA, Monounsaturated fatty acids;
SD, Standard Deviation;
CV, Coefficient of variation [symbol]
indicates data missing or illegible when filed

As depicted in Table 6, Tukey-adjusted multiple genotype comparison tests at the SCD g.23881050T>C SNP locus confirmed significant differences where the homozygous TT genotype had the highest DHA (11.00±2.34 mg/100 g), IMF (5.43±0.516%), and DPA (27.1±3.26 mg/100 g) compared to the CC genotype with the lowest DHA (7.00±2.11 mg/100 g), IMF (3.98±0.312%), and DPA (17.9±6.81 mg/100 g). The heterozygous genotype CT had intermediate DPA (7.64±2.09 mg/100 g), IMF (4.39±0.287%), and DPA (19.4±6.74 mg/100 g) that were in-between the highest and lowest values (Table 6). There were many more significant genotype variations at the FASN g.12323864A>G SNP mutation that was associated with FMP, ALA, MUFA, SFA, C18:2n-6, C18:1n-9, C18:0, and C16:0, in which the homozygous genotype GG had the highest values compared to the lowest values in AA genotype for all variables, with the exception of C18:2n-6 that was lowest in the heterozygous GA genotype (Table 6). In contrast, at the FABP4 g.62829478A>G SNP locus, only IMF variation tended towards significance between the genotypes (p<0.06).

TABLE 6
Tukey-adjusted multiple comparisons between SNP mutations and FMP, IMF,
and fatty acids in TAW lambs ^{#}.
Multiple Genotype Comparisons

| SNP Locus | Variable | Mean ± SE | Genotypes | Difference ± SE | p-Value |
|---|---|---|---|---|---|
| SCD | DHA (C22:6n-3) | | | | |
| g.23881050T > C | (mg/100 g) | | | | |
| | CC | 7.00 ± 2.11 | CC vs. CT | −0.639 ± 0.834 | 0.7247 |
| | CT | 7.64 ± 2.09 | CC vs. TT | −3.998 ± 1.334 | 0.0105 * |
| | TT | 11.00 ± 2.34 | CT vs. TT | −3.359 ± 1.235 | 0.0223 * |
| | IMF (%) | | | | |
| | CC | 3.98 ± 0.312 | CC vs. CT | −0.407 ± 0.323 | 0.4224 |
| | CT | 4.39 ± 0.287 | CC vs. TT | −1.446 ± 0.532 | 0.0222 * |
| | TT | 5.43 ± 0.516 | CT vs. TT | −1.038 ± 0.502 | 0.1041 |
| | DPA (C22:5n-3) | | | | |
| | (mg/100 g) | | | | |
| | CC | 17.9 ± 6.81 | CC vs. CT | −1.56 ± 2.65 | 0.8270 |
| | CT | 19.4 ± 6.74 | CC vs. TT | −9.19 ± 4.25 | 0.0850 |
| | TT | 37.1 ± 3.26 | CT vs. TT | −7.63 ± 3.93 | 0.0356 * |
| FASN | FMP (° C.) | | | | |
| g.12323864A > G | GG | 34.2 ± 0.4 | GG vs. GA | 0.81 ± 0.64 | 0.4201 |
| | GA | 33.4 ± 0.3 | GG vs. AA | 2.98 ± 1.61 | 0.0536 * |
| | AA | 31.5 ± 1.5 | GA vs. AA | 2.16 ± 1.60 | 0.3685 |
| | ALA (C18:3n-3) | | | | |
| | (mg/100 g) | | | | |
| | GG | 188.7 ± 67.6 | GG vs. GA | 114.7 ± 39.9 | 0.0149 * |
| | GA | 74.0 ± 66.7 | GG vs. AA | 147.2 ± 100.1 | 0.3115 |
| | AA | 41.5 ± 113.7 | GA vs. AA | 32.6 ± 99.8 | 0.9430 |
| | MUFA | | | | |
| | (mg/100 g) | | | | |
| | GG | 4524 ± 1384 | GG vs. GA | 2617 ± 867 | 0.0099 ** |
| | GA | 1907 ± 1361 | GG vs. AA | 3089 ± 2175 | 0.3363 |
| | AA | 1436 ± 2415 | GA vs. AA | 472 ± 2168 | 0.9742 |
| | SFA | | | | |
| | (mg/100 g) | | | | |
| | GG | 5479 ± 1715 | GG vs. GA | 3270 ± 1121 | 0.0132 * |
| | GA | 2208 ± 1684 | GG vs. AA | 4162 ± 2812 | 0.3068 |
| | AA | 1317 ± 3086 | GA vs. AA | 892 ± 2803 | 0.9458 |
| | C18:2n-6 | | | | |
| | (mg/100 g) | | | | |
| | GG | 281 ± 84.8 | GG vs. GA | 142.5 ± 52.2 | 0.0216 * |
| | GA | 139 ± 83.4 | GG vs. AA | 105.5 ± 130.8 | 0.6988 |
| | AA | 175 ± 146.4 | GA vs. AA | −36.7 ± 130.4 | 0.9573 |
| | C16:0 | | | | |
| | (mg/100 g) | | | | |
| | GG | 2539 ± 800 | GG vs. GA | 1475 ± 518 | 0.0158 * |
| | GA | 1063 ± 786 | GG vs. AA | 1826 ± 1298 | 0.3433 |
| | AA | 713 ± 1429 | GA vs. AA | 350 ± 1294 | 0.9604 |
| | C18:0 | | | | |
| | (mg/100 g) | | | | |
| | GG | 2227 ± 698 | GG vs. GA | 1419 ± 441 | 0.0056 ** |
| | GA | 809 ± 646 | GG vs. AA | 1711 ± 1106 | 0.2756 |
| | AA | 516 ± 1205 | GA vs. AA | 292 ± 1102 | 0.9620 |
| | C18:1n-9 | | | | |
| | (mg/100 g) | | | | |
| | GG | 3589 ± 1075 | GG vs. GA | 2103 ± 679 | 0.0080 ** |
| | GA | 1486 ± 1060 | GG vs. AA | 2353 ± 1704 | 0.3566 |
| | AA | 1236 ± 1892 | GA vs. AA | 250 ± 1698 | 0.9882 |
| FABP4 | IMF (%) | | | | |
| g.62829478A > T | A | 4.57 ± 0.39 | A vs. AA | 0.07 ± 0.344 | 0.0556 |
| | AA | 3.92 ± 0.39 | | | |

[symbol]

p < 0.03, [symbol]
p < 0.01;
SFA, Saturated fatty acids; MUFA, Monosaturated fatty acids; SD, Standard Deviation; CV, Coefficient of variation. [symbol]
indicates data missing or illegible when filed

The data shown here provides insights into the shared genetic control of the FMP, IMF content, and health-beneficial omega-3 long-chain fatty acid composition traits that are helpful in designing breeding strategies to genetically improve meat-eating quality traits in TAW lambs while they are still alive. The early decision making utilizing this minimally invasive longissimus dorsi thoracis et lumborum muscle biopsy sampling technique for directly quantifying the genetic worth of live lambs advantageously overcomes the problem of waiting to collect meat quality data after slaughter when selection decisions about the live animal are already too late. The identified SNPs of these lipid metabolism genes can also be used for breed-specific identification and marker-assisted selection of TAW sheep for high-end meat-eating quality. NGS of the FABP4, FASN, and SCD genes also provides data demonstrating their roles in fatty acid metabolism unique to the TAW breed.

### Example 2: Single Nucleotide Polymorphism (SNP)-Based DNA Marker Identity Test

Based on the findings in Example 1, the inventors further developed a SNP-based DNA marker identity test as a diagnostic measure for determining true-to-type Tattykeel Australian White Lambs (TAWL) characterized by high FMP, IMF and n-3 LC-PUFA. The following procedures were carried out to create the DNA marker identity test. The extracted genomic DNA (gDNA) from TAWLs were diluted to 100 ng/uL. The concentration of gDNA was checked in Quantifluor dsDNA System (Promega, USA) for accuracy.

PCR amplification assay was performed using the Platinum™ SuperFi™ II PCR Master Mix (Thermofisher Scientific, Australia). The amplification PCR reaction assay was performed in a total volume of 10 μL containing 5 μL of 2X Platinum™ SuperFi™ II PCR Master Mix, 4 μL of 10 μM of each forward inner/reverse inner and forward outer/reverser outer tetra-primers amplification refractory mutation system (tetra-primer ARMS-PCR:IDT, Singapore), and 0.5 uL of 100 ng of gDNA template. PCR was performed in a SimpliAmp™ Thermal Cycler (Thermofisher Scientific, Melbourne, Australia), in a 3-steps protocol using the following conditions: 98° C. initial denaturation for 1 minute (1 cycle); 98° C. denaturation for 15 seconds; 65° C. annealing for 15 seconds: 72° C. extension for 9 minutes; 72° C. final extension for 9 minutes; for 35 cycles and hold at 4° C. Changing annealing temperature from between 50-62 was not efficient to improve PCR products. Using 100 uM of forward outer primer (a) improved the PCR product, and was helpful in genotyping different sheep breeds (FIG. 2). PCR success was checked in 1.5% agarose gel electrophoresis, set for 2 hours, as depicted in FIGS. 1-4.

TABLE 1
Name and alleles of SNPs, primers and amplicon size used for tetra-primer ARMS-
PCR genotyping in Tattykeel Australian White Lamb.

| | | | | Amplicon | |
|---|---|---|---|---|---|
| SNP | Primer | Sequence (5′-3′) | Allele | size (bp) | SEQ ID NO |
| g.23881050T > C | Forward | GTGCTCGGTGGGCATTCCCACCTCGG | T | 199 | SEQ ID NO: 1 |
| | inner | | | | |
| | Reverse | TTCCCCTTCCCAGGCCAGACTCATAGA | C | 235 | SEQ ID NO: 2 |
| | inner | | | | |
| | Forward | TGCTCGGTGGGCATT | C/T | 85 | SEQ ID NO: 3 |
| | outer (a) | | | | |
| | Reverse | CTCCCATCCCTGGTCTTTA | | 85 | SEQ ID NO: 4 |
| | outer (a) | | | | |
| | Forward | TCCACACCCATCCTGCTCCAGCCACTCT | C/T | 378 | SEQ ID NO: 5 |
| | outer (b) | | | | |
| | Reverse | TGCAGCTGTCTGCAAGCACCCCATACCC | | 378 | SEQ ID NO: 6 |
| | outer (b) | | | | |

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1.-43. (canceled)

44. A method for identifying an ovine animal having desirable omega-3 long-chain polyunsaturated fatty acids (n-3 LC-PUFA), intramuscular fat (IMF), fat melting point (FMP) or any combination thereof, as compared to the general population of animals of that species, said method comprising:
(a) obtaining single nucleotide polymorphism (SNP) genotype data for the ovine animal at one or more SNP loci selected from:
(i) SCD g.23881050T>C or a SNP in linkage disequilibrium with SCD g.23881050T>C having an r² value of ≥0.7; and/or
(ii) FASN g.12323864A>G or a SNP in linkage disequilibrium with FASN g.12323864A>G having an r² value of ≥0.7; and/or
(iii) FABP4 g.62829478A>T or a SNP in linkage disequilibrium with FABP4 g.62829478A>T having an r² value of ≥0.7; and
(b) determining that the ovine animal has or will have desirable n-3 LC-PUFA, IMF, FMP or any combination thereof, as compared to the general population of animals of that species, if the animal possesses:
(i) a TT genotype in SCD g.23881050T>C or a genotype in linkage disequilibrium with said TT genotype, and/or
(ii) a GG genotype in FASN g.12323864A>G or a genotype in linkage disequilibrium with said GG genotype, and/or
(iii) an AA genotype in FABP4 g.62829478A>T or a genotype in linkage disequilibrium with said AA genotype.

45. The method of claim 44, comprising at least one of (i)-(iii):
wherein (i) comprises:
(a) obtaining SNP genotype data for the ovine animal at SNP locus SCD g.23881050T>C or a SNP in linkage disequilibrium with SCD g.23881050T>C having an r² value of ≥0.7, and
determining that the ovine animal has or will have desirable n-3 LC-PUFA, IMF, FMP or any combination thereof, as compared to the general population of animals of that species, if the animal possesses a TT genotype in SCD g.23881050T>C, or a genotype in linkage disequilibrium with said TT genotype; and/or
(b) obtaining SNP genotype data for the ovine animal at SCD g.23881050T>C; and
determining that the ovine animal has or will have desirable n-3 LC-PUFA, IMF, FMP or any combination thereof, as compared to the general population of animals of that species, if the animal possesses a TT genotype in SCD g.23881050T>C;
wherein (ii) comprises:
(a) obtaining SNP genotype data for the ovine animal at SNP locus FASN g.12323864A>G or a SNP in linkage disequilibrium with FASN g.12323864A>G having an r² value of ≥0.7, and determining that the ovine animal has or will have desirable n-3 LC-PUFA, IMF, FMP or any combination thereof, as compared to the general population of animals of that species, if the animal possesses a GG genotype in FASN g.12323864A>G, or a genotype in linkage disequilibrium with said GG genotype; and/or
(b) obtaining SNP genotype data for the ovine animal at SNP locus FASN g.12323864A>G; and determining that the ovine animal has or will have desirable n-3 LC-PUFA, IMF, FMP or any combination thereof, as compared to the general population of animals of that species, if the animal possesses a GG genotype in FASN g.12323864A>G; and
wherein (iii) comprises:
(a) obtaining SNP genotype data for the ovine animal at SNP locus FABP4 g.62829478A>T or a SNP in linkage disequilibrium with FABP4 g.62829478A>T having an r² value of ≥0.7, and determining that the ovine animal has or will have desirable n-3 LC-PUFA, IMF, FMP or any combination thereof, as compared to the general population of animals of that species, if the animal possesses a AA genotype in FABP4 g.62829478A>T, or a genotype in linkage disequilibrium with said AA genotype; and/or
(b) obtaining SNP genotype data for the ovine animal at SNP locus FASN g.12323864A>G; and determining that the ovine animal has or will have desirable n-3 LC-PUFA, IMF, FMP or any combination thereof, as compared to the general population of animals of that species, if the animal possesses a AA genotype in FABP4 g.62829478A>T.

46.-50. (canceled)

51. The method of claim 44, further comprising selecting and/or breeding from an ovine animal identified as having desirable n-3 LC-PUFA, IMF, FMP or any combination thereof, as compared to the general population of animals of that species, based on SNP genotype.

52. A method for determining if an ovine animal or part thereof is an Australian White Lamb or part thereof, said method comprising:
(a) obtaining single nucleotide polymorphism (SNP) genotype data for the ovine animal at one or more SNP loci selected from:
(i) SCD g.23881050T>C or a SNP in linkage disequilibrium with SCD g.23881050T>C having an r² value of ≥0.7;
(ii) FASN g.12323864A>G or a SNP in linkage disequilibrium with FASN g.12323864A>G having an r² value of ≥0.7; and/or
(iii) FABP4 g.62829478A>T or a SNP in linkage disequilibrium with FABP4 g.62829478A>T having an r² value of ≥0.7; and
(b) determining that the ovine animal or part thereof is an Australian White Lamb or part thereof if the animal possesses:
(i) a TT genotype in SCD g.23881050T>C or a genotype in linkage disequilibrium with said TT genotype;
(ii) a GG genotype in FASN g.12323864A>G or a genotype in linkage disequilibrium with said GG genotype; and/or
(iii) an AA genotype in FABP4 g.62829478A>T or a genotype in linkage disequilibrium with said AA genotype.

53. The method of claim 52, comprising at least one of (i)-(iii):
wherein (i) comprises:
(a) obtaining SNP genotype data for the ovine animal at SNP locus SCD g.23881050T>C or a SNP in linkage disequilibrium with SCD g.23881050T>C having an r² value of ≥0.7, and
determining that the ovine animal or part thereof is an Australian White Lamb or part thereof if the animal possesses a TT genotype in SCD g.23881050T>C, or a genotype in linkage disequilibrium with said TT genotype; and
wherein (ii) comprises:
(a) obtaining SNP genotype data for the ovine animal at SNP locus FASN g.12323864A>G or a SNP in linkage disequilibrium with FASN g.12323864A>G having an r² value of ≥0.7, and determining that the ovine animal or part thereof is an Australian White Lamb or part thereof if the animal possesses a GG genotype in FASN g.12323864A>G, or a genotype in linkage disequilibrium with said GG genotype; and/or
(b) obtaining SNP genotype data for the ovine animal at SNP locus FASN g.12323864A>G; and determining that the ovine animal or part thereof is an Australian White Lamb or part thereof if the animal possesses a GG genotype in FASN g.12323864A>G; and/or
wherein (iii) comprises:
(a) obtaining SNP genotype data for the ovine animal at SNP locus FABP4 g.62829478A>T or a SNP in linkage disequilibrium with FABP4 g.62829478A>T having an r² value of ≥0.7, and determining that the ovine animal or part thereof is an Australian White Lamb or part thereof if the animal possesses a AA genotype in FABP4 g.62829478A>T, or a genotype in linkage disequilibrium with said AA genotype; and/or
(b) obtaining SNP genotype data for the ovine animal at SNP locus FASN g.12323864A>G; and determining that the ovine animal or part thereof is an Australian White Lamb or part thereof if the animal possesses a AA genotype in FABP4 g.62829478A>T.

54.-58. (canceled)

59. The method of claim 52, further comprising selecting and/or breeding from an ovine animal or part thereof identified as being an Australian White Lamb based on SNP genotype.

60. The method of claim 44, further comprising generating the SNP genotype data from a nucleic acid sample from the ovine animal.

61. The method of claim 60, wherein the nucleic acid sample comprises at least one of the following features:
(i) is prepared from tissue taken from the longissimus dorsi thoracis et lumborum of the animal;
(b) is prepared from reproductive material of an animal selected from semen, an oocyte or an embryo;
(c) is prepared from blood or a blood fraction; and
(d) is DNA;
optionally, wherein SNP alleles for each ovine animal are detected by sequencing a subsequence of the gene comprising each SNP and/or wherein SNP alleles are detected by one or more DNA hybridisation assays using polynucleotides which permit different alleles of a SNP to be distinguished from one another.

62.-64. (canceled)

65. The method of claim 44, wherein:
(a) the SNP genotype data is generated using one or more assays selected from the group consisting of: a DNA amplification assay, a DNA hybridisation assay, DNA sequencing, denaturing high-performance liquid chromatography (DHPLC) or a combination thereof; and/or
(b) SNP alleles are detected by one or more DNA amplification assays using polynucleotides which permit different alleles of a SNP to be distinguished from one another;
and/or, optionally:
(c) wherein the DNA amplification assay is polymerase chain reaction (PCR);
(d) wherein the PCR is amplification-refractory mutation system (ARMS) PCR.

66.-70. (canceled)

71. A test kit comprising
a plurality of oligonucleotide primers and/or probes configured to discriminate between alleles at one or more SNP loci in an ovine animal, wherein the SNP loci are selected from
(i) SCD g.23881050T>C or a SNP in linkage disequilibrium with SCD g.23881050T>C having an r² value of ≥0.7,
(ii) FASN g.12323864A>G or a SNP in linkage disequilibrium with FASN g.12323864A>G having an r² value of ≥0.7, and/or
(iii) FABP4 g.62829478A>T or a SNP in linkage disequilibrium with FABP4 g.62829478A>T having an r² value of ≥0.7.

72. The test kit according to claim 71, comprising:
one or more oligonucleotide primers and/or probes configured to discriminate between allele C and allele T at SNP locus SCD g.23881050T>C; and/or
one or more oligonucleotide primers and/or probes configured to discriminate between allele G and allele G at SNP locus FASN g.12323864A>G; and/or
one or more oligonucleotide primers and/or probes configured to discriminate between allele A and allele G at SNP locus FASN g.12323864A>G;
optionally, wherein the oligonucleotide primers are configured to discriminate between alleles by amplification-refractory mutation system polymerase chain reaction (ARMS PCR).

73. (canceled)

74. The test kit of claim 72, wherein the oligonucleotide primers configured to discriminate between allele C and allele T at SNP locus SCD g.23881050T>C by ARMS PCR comprise:
a primer comprising the sequence set forth in SEQ ID NO: 1 or a sequence which is substantially identical thereto (designated “SCD g.23881050T>C_Forward inner”);
a primer comprising the sequence set forth in SEQ ID NO: 2 or a sequence which is substantially identical thereto (designated “SCD g.23881050T>C_Reverse inner”);
a primer comprising the sequence set forth in SEQ ID NO: 3 or a sequence which is substantially identical thereto (designated “SCD g.23881050T>C_Forward outer (a)”);
a primer comprising the sequence set forth in SEQ ID NO: 4 or a sequence which is substantially identical thereto (designated “SCD g.23881050T>C_Reverse outer (a)”);
a primer comprising the sequence set forth in SEQ ID NO: 5 or a sequence which is substantially identical thereto (designated “SCD g.23881050T>C_Forward outer (b)”); and
a primer comprising the sequence set forth in SEQ ID NO: 6 or a sequence which is substantially identical thereto (designated “SCD g.23881050T>C_Reverse outer (b)”).

75. The test kit of claim 72, wherein the oligonucleotide primers configured to discriminate between allele C and allele T at SNP locus SCD g.23881050T>C by ARMS PCR are:
SCD g.23881050T>C_Forward inner (SEQ ID NO: 1);
SCD g.23881050T>C_Reverse inner (SEQ ID NO: 2);
SCD g.23881050T>C_Forward outer (a) (SEQ ID NO: 3);
SCD g.23881050T>C_Reverse outer (a) (SEQ ID NO: 4);
SCD g.23881050T>C_Forward outer (b) (SEQ ID NO: 5); and
SCD g.23881050T>C_Reverse outer (b) (SEQ ID NO: 6).

76. The test kit of claim 71, comprising one or more or each of the following:
at least one DNA polymerase enzyme;
dinucleotide triphosphates (dNTPs);
a magnesium salt; and
a buffer.

77. The test kit of claim 71, for detecting an ovine animal having desirable omega-3 long-chain polyunsaturated fatty acids (n-3 LC-PUFA), intramuscular fat (IMF), fat melting point (FMP) or any combination thereof, as compared to the general population of animals of that species.

78. The test kit of claim 71 when used in a method for identifying an ovine animal having desirable omega-3 long-chain polyunsaturated fatty acids (n-3 LC-PUFA), intramuscular fat (IMF), fat melting point (FMP) or any combination thereof, as compared to the general population of animals of that species; or a method for determining if an ovine animal or part thereof is an Australian White Lamb or part thereof; either method comprising:
(a) obtaining single nucleotide polymorphism (SNP) genotype data for the ovine animal at one or more SNP loci selected from:
(i) SCD g.23881050T>C or a SNP in linkage disequilibrium with SCD g.23881050T>C having an r² value of ≥0.7; and/or
(ii) FASN g.12323864A>G or a SNP in linkage disequilibrium with FASN g.12323864 A>G having an r² value of ≥0.7; and/or
(iii) FABP4 g.62829478A>T or a SNP in linkage disequilibrium with FABP4 g.62829478 A>T having an r² value of ≥0.7; and
(b) determining that the ovine animal has or will have desirable n-3 LC-PUFA, IMF, FMP or any combination thereof, as compared to the general population of animals of that species, if the animal possesses:
(i) a TT genotype in SCD g.23881050T>C or a genotype in linkage disequilibrium with said TT genotype, and/or
(ii) a GG genotype in FASN g.12323864A>G or a genotype in linkage disequilibrium with said GG genotype, and/or
(iii) an AA genotype in FABP4 g.62829478A>T or a genotype in linkage disequilibrium with said AA genotype.

79.-80. (canceled)

81. A method for producing reproductive or regenerative material from an ovine animal determined as having desirable omega-3 long-chain polyunsaturated fatty acids (n-3 LC-PUFA), intramuscular fat (IMF), fat melting point (FMP) or any combination thereof, as compared to the general population of animals of that species, said method comprising:
(i) performing the method according to claim 44 on one or more animals to thereby identify an ovine animal having desirable n-3 LC-PUFA, IMF, FMP or any combination thereof, as compared to the general population of animals of that species;
(ii) selecting the animal identified in (i) as having desirable n-3 LC-PUFA, IMF, FMP or any combination thereof, as compared to the general population of animals of that species; and
(iii) obtaining reproductive or regenerative material from the selected animals.

82. Reproductive or regenerative material from an ovine animal produced according to the method of claim 81.

83. A method of breeding an ovine animal having desirable omega-3 long-chain polyunsaturated fatty acids (n-3 LC-PUFA), intramuscular fat (IMF), fat melting point (FMP) or any combination thereof, as compared to the general population of animals of that species, said method comprising:
(i) performing the method according to claim 44 on one or more animals to thereby identify one or more ovine animals having desirable n-3 LC-PUFA, IMF, FMP or any combination thereof, as compared to the general population of animals of that species;
(ii) selecting one or more animals identified in (i) as having desirable n-3 LC-PUFA, IMF, FMP or any combination thereof, as compared to the general population of animals of that species;
(iii) breeding from the selected animals to produce one or more offspring therefrom.

84. An ovine animal bred according to the method of claim 83.

85. A method for improving meat eating quality in a population of ovine animals, comprising:
(i) performing the method according to claim 44 on one or more animals in the population to thereby identify one or more ovine animals having desirable omega-3 long-chain polyunsaturated fatty acids (n-3 LC-PUFA), intramuscular fat (IMF), fat melting point (FMP) or any combination thereof, as compared to the general population of animals of that species;
(ii) selecting one or more animals identified in (i) as having desirable n-3 LC-PUFA, IMF, FMP or any combination thereof, as compared to the general population of animals of that species;
(iii) obtaining reproductive or regenerative material from the selected animals; and
(iv) producing one or more offspring or one or more generations of animals from the reproductive or regenerative material.
